(19) United States
(12) Patent Application Publication
(10) Pub. No.: US 2022/0364069 A1
(43) Pub. Date: Nov. 17, 2022

(54) METHOD FOR THE PRODUCTION OF AN ENZYMATIC COMPOSITION COMPRISING A RECOMBINANT ENDOPEPTIDASE

(71) Applicant: NEMYSIS LIMITED, Dublin 7 (IE)

(72) Inventors: Anna Taravella, Saronno (VA) (IT); Giacomo Carenzi, Busto Arsizio (VA) (IT); Alessandro Sigurta', Saronno (VA) (IT); Linda Cavaletti, Rovellasca (CO) (IT)

(73) Assignee: NEMYSIS LIMITED, Dublin 7 (IE)

(21) Appl. No.: 17/626,822

(22) PCT Filed: Jul. 8, 2020
(86) PCT No.: PCT/EP2020/069297

§ 371 Date: Jan. 13, 2022

Related U.S. Application Data
(60) Provisional application No. 62/878,369, filed on Jul. 25, 2019.

(30) Foreign Application Priority Data
Jul. 25, 2019 (IT) 102019000012942

(51) Int. Cl. C12N 9/52; C12N 1/20; C12N 15/76; A61K 38/48; A23L 29/00; A21D 8/04; A23L 33/10; A23L 33/00

(52) U.S. Cl. CPC C12N 9/52; C12N 1/20; C12N 15/76; C12Y 304/21026; A61K 38/48; A23L 29/06; A21D 8/042; A23L 33/10; A23L 33/40; C12N 2800/101; A23V 2002/00

(57) ABSTRACT

The present invention is directed to a method for manufacturing an enzyme preparation, comprising at least one recombinant Actinoallomurus endopeptidase with glutenase activity, at high yields and suitable for human use. The invention is further directed to a recombinant expression vector for expressing the recombinant endopeptidase(s) of interest, and to a S. lividans host cell comprising said vector. Moreover, the present invention is directed the enzyme preparation obtained by said method, to formulations of the same and to clinical uses thereof.

Sequence listing ASCII file Sequence_Corrected_v2.txt, created Jul. 5, 2022 and of size of 39,000 bytes is incorporated herein by reference.

### Field of the Invention

Celiac Disease (CD) is a chronic autoimmune enteropathy triggered by gluten¹. Gluten is a heterogeneous mixture of insoluble proteins, consisting of gliadins and glutenins, present in wheat, rye and barley cereals²; gluten proteins are largely inaccessible to human proteases of the gastrointestinal tract, therefore large proline/glutamine rich peptides can reach the small intestine and trigger both humoral and T-cell mediated adaptive immune responses in patients with CD. To date several T-cell stimulatory peptides, resistant to gastrointestinal digestion, have been identified either in gliadins and glutenin proteins; among these, a 33-mer peptide from α-gliadin having sequence LQLQPFPQPQLPYPQPQLPYPQPQLPYPQPQPF (SEQ ID NO: 12) is currently considered to be the most immunogenic peptide, as it carries multiple copies of epitopes that are immunogenic in patients with CD⁵.

Life-long adherence to a strict gluten-free diet (GFD) is the current treatment for CD patients⁶. However, total gluten avoidance is pragmatically impossible and novel therapies have been intensively investigated⁷, leading to the development of novel enzymes, termed “glutenases”, which are capable of digesting gluten and toxic fragments of gluten in the gastrointestinal environment.

WO2013083338 discloses a group of novel glutenases, i.e. endopeptidases with glutenase activity, identified by inventors of the present application in acidophilic actinomycete Actinoallomurus A8. Said Actinoallomurus endopeptidases are very rapid and efficient in degrading gluten peptides into non-toxic peptides, being active at the whole pH range of the gastric and intestinal environment (enzymatic activity is shown in the range of pH 3-6 with optimum at pH 5) and being moreover resistant to degradation by gastrointestinal endogenous enzymes. This makes these novel Actinoallomurus endopeptidases very suitable for being used in the treatment and/or prevention of CD and CD-associated disorders. Among said Actinoallomurus endopeptidases disclosed in WO2013083338, endopeptidase 40 (E40) has been found to be of particular interest. E40 native protein consists of 398 amino acid residues (see SEQ ID NO: 3), belonging to the serine-carboxyl peptidase S53 family with the catalytic triad formed by aspartic acid, glutamic acid and serine at positions 156, 160 and 329, respectively. The N-terminal signal peptide has been identified by signalP 4.1 server analysis between positions 27 and 28, and it has been predicted that the mature form started from position 74, resulting in a 32.5 kDa mature enzyme, as expected. The mature form of native E40 is a polypeptide of sequence SEQ ID NO: 1.

There is a strong interest in developing efficient and inexpensive methods of manufacturing said Actinoallomurus endopeptidases having glutenase activity, in particular E40 of sequence comprising or consisting of SEQ ID NO: 1.

E40 is hereafter used to identify an endopeptidase of sequence comprising SEQ ID NO: 1, i.e. an endopeptidase of sequence consisting of SEQ ID NO: 1 or a derivative thereof having sequence comprising SEQ ID NO:1; an exemplary derivative of E40 is a tagged E40, such as an endopeptidase having sequence comprising or consisting of SEQ ID NO: 2.

Recombinant DNA (rDNA) technology offers a very potent set of technical platforms for the controlled and scalable production of polypeptides of interest by relatively inexpensive procedures. Recombinant proteins are today obtained in Escherichia coli, Saccharomyces cerevisiae, in insect, hamster and mammalian cells. There is however a demand for improving the production of big quantities of recombinant proteins; furthermore, there are very strict requirements to be fulfilled when proteins are produced for human use, for instance for use as food supplements and/or as medicaments in the prevention and/or treatment of human diseases.

The present invention is thus aimed at providing an improved method for efficient and inexpensive manufacturing of an enzyme preparation that comprises at least one of the Actinoallomurus endopeptidases disclosed in WO2013083338, as recombinant proteins, said enzyme preparation being suitable for human use, optionally further to proper formulation.

Streptomycetes are regarded as a safe source of proteins for human alimentary use. Two examples of food enzymes sourced from Streptomyces spp are: glucose isomerases used for fructose syrup production¹², and the widely exploited transglutaminase from S. mobaraensis, used in food industry for its properties in improving the texture and overall quality of final food products, such as processed meat and fish products, as well as diary and baked food¹³.

The present inventors have found that Streptomyces lividans is a particularly advantageous host cell to be employed in the improved method of the invention.

The present invention therefore provides a method for the production of an enzyme preparation, that comprises at least one recombinant Actinoallomurus endopeptidase with glutenase activity, in S. lividans host cells, the method being characterized by a phase of culturing said host cells, expressing the recombinant Actinoallomurus endopeptidase(s) of interest, under fermentation conditions, followed by a phase of recovery of the supernatant of the host cell's culture medium, comprising the produced recombinant endopeptidase(s); preferably said phase of recovery is followed by a phase of purification from said supernatant of a final enzyme preparation comprising the recombinant Actinoallomurus endopeptidase(s) with glutenase activity of interest.

S. lividans cells are known to be efficient host cells for the production of recombinant proteins, since recombinant proteins expressed by said cells can be directly secreted and released in the culture medium. However, different proteins are obtained at very different yields in S. lividans, this result being unpredictable¹². Moreover, S. lividans cells produce high levels of secondary metabolites, in particular antibiotics¹², thus jeopardizing the possibility of establishing S. lividans as a proper cell factory for the manufacturing of enzymatic preparations aimed to supplement physiological digestive enzymes.

The method of the invention, returns an enzyme preparation that comprises high amounts of the desired recombinant glutenase. Surprisingly, despite culturing the host cells under fermentation conditions, the enzymatic preparation obtained by the method of the invention is substantially free of of potential harmful secondary metabolites released from S. lividans, such as e.g. antibiotics, which would be regulatory unacceptable for human intake within food, food supplements or pharmaceutical formulations; the enzyme preparation obtained by the method of the invention is suitable for human use (optionally further to proper formulation of the same).

### BRIEF DESCRIPTION OF THE INVENTION

The present invention is directed to a method for manufacturing an enzymatic preparation comprising at least one recombinant Actinoallomurus endopeptidase with glutenase activity, characterized in that a batch of S. lividans host cells capable of expressing a recombinant Actinoallomurus endopeptidase is cultured under fermentation conditions in a medium comprising at least 30% (wt/vol) sucrose, the enzymatic preparation being then recovered and purified from the supernatant of the host cells culture. The enzymatic preparation is obtained at high yields by the method of the invention and it is substantially free of antibiotics, being thus suitable for human use. The term “substantially free” of antibiotics means that no antibiotics are detected in the enzymatic preparation, either in microbiological and quantitative HPLC assays.

The present invention is further directed to said enzyme preparation obtained by said method, to formulations of the enzyme preparation, suitable for human use, to a recombinant expression vector bearing a nucleic acid encoding for the recombinant Actinoallomurus endopeptidase(s) of interest and to a S. lividans host cell comprising said recombinant expression vector and stably expressing said recombinant Actinoallomurus endopeptidase(s). Moreover, the present invention is directed to clinical uses of the enzyme preparation and formulations thereof. Preferably, the at least one Actinoallomurus endopeptidase of interest, having glutenase activity, is E40 or a derivative thereof, having sequence comprising SEQ ID NO: 1.

### BRIEF DESCRIPTION OF FIGURES

FIG. 1. Activity of Actinoallomurus A8 culture supernatant fraction containing the native E40. A: relative activity toward the substrate sucAlaAlaProPhe-AMC (sucAAPF-AMC) at various pH values (the optimum —100% activity— is at pH 5). B: Gliadin digestion after 2 hours of incubation at 37° C., pH 3. Gel stained with Coomassie R250 brilliant blue; Lane 1: gliadin alone, lane 2: gliadin+E40-containing supernatant fraction.

FIG. 2. map of vector pIJ86 bearing the E40 coding gene (pIJ86/e40 recombinant expression vector)

FIG. 3. Recombinant E40 (SEQ ID NO: 2) obtained by S. lividans TK24/p1J86/e40 submerged fermentation, at flask scale (panels A-C) or in a 15 L bioreactor, followed by purification (panels D-E). A: activity toward the substrate sucAAPF-AMC at pH 5 of supernatant samples taken at different fermentation times (♦72, ▴96, ●168, ▪192 fermentation hours) from the producing S. lividans TK24/p1J86/E40 (E40, continuous line) and absence of activity of supernatant samples taken from the pIJ86-empty control strain (ctr, dashed lines). Activity is shown as relative fluorescent units (rfu, Y axis) produced in time (X axis). B: relative activity (%) showed by the E40 supernatant sample at 168 hours of fermentation, at different pH: the optimum (100% activity) is at pH 5. C: zymography of the 168 h E40 supernatant sample at pH 5, using the same substrate as in panel A and visualized under UV light. D: profile of an enzyme preparation comprising recombinant E40 purified from the 15 L fermentation; lane 1: final enzyme preparation comprising recombinant E40 stained with Coomassie (the arrow indicates recombinant E40), lane 2: zymography as in C, lane 3: band of gelatin hydrolysis after 2 hours of digestion at 37° C. and pH 5, visualized after Coomassie staining of the gelatin substrate. E: relative activity (%) of recombinant E40 in the purified enzyme preparation at different pH values (from 2 to 8), evaluated with the substrate suc-Ala-Ala-Pro-Phe-pNA.

FIG. 4. Activity of recombinant E40 evaluated in the absence or presence of digestive proteases pepsin (P) (panels A-C) at pH 4, 4.5 and 5, and trypsin (T) (panels D-E, E′) at pH 6 and 7. Reactions started by addition of the substrate to enzyme preparation samples comprising the E40 enzyme alone (line \textcircled{1}), enzyme and P (line \textcircled{2})enzyme and T (line \textcircled{4}), and by addition of P or T alone (lines \textcircled{9} and (\textcircled{5} respectively). Each reaction condition was tested in duplicate, error bars represent the standard deviation. Panel E′ is a magnification of E. X axis: incubation time (minutes), Y axis: absorbance units read at 410 nm. (R² values are 0.9948, 0.9976, 0.9978, 0.9639, 0.9994 for E40 alone and 0.993, 0.9975, 0.9982: 0.9661, 0.9994 for E40+P/T for pH 4, 4.5, 5, 6 and 7, respectively).

FIG. 5. LC/MS profile of digestion of 33-mer peptide by recombinant E40 at pH 5 and 37° C., with E40 in a molar ratio 1:48 to 33-mer peptide. A: full-length peptide alone (triply charged ion [(M⁺3H⁺)³⁺=1304.68]). a=14.75;1304.63. B-E: time-course of 33-mer cleavage by E40 activity. b=13.07,1010.27; c=14.84,1304.46; d=14.99,1304.50; e=15.13,1304.14; f=7.80,842.11; g=8.02,842.34; h=8.24,842.36; i=11.62,244.16; l=11.84,1086.38; m=12.38,745.13; n=13.17,956.23; o=7.52, 842.38; p=11.59,1086.51; q=12.12,745.22; r=6.66,842.37; s=11.45,1086.48; t=11.93,745.23. Peptide fragments formed during digestion are indicated by arrows. F: schematic visualization of E40 cleavage sites deduced from the final residual peptides.

FIG. 6. HPLC analysis of gliadin incubated with recombinant E40 at different incubation time, up to 240 min. At time point 0, gliadin is eluted according to hydrophobicity in ω-, α-, and γ-gliadin.

FIG. 7. A: Recognition of E40-treated gliadin in gliadin reactive T-cell lines obtained from jejunal biopsies of 5 different celiac subjects (samples A-H). Positive controls: untreated gliadin digests (samples I and L) and phytohemagglutinin (PHA). Each panel is a representative experiment out of three done for each T-cell line. B: Immunostimulatory activity of gliadin purified from hexaploidy wheat on human intestinal T cells, after digestion with E40, shown as percent of T-cell response to gliadin digests treated with the sole E40 (samples A-B), or with E40 in the presence of gastrointestinal proteases (samples C-H), calculated on the E40-untreated gliadin digests (sample I), at the conditions specified in Table 1. The enzymatic digests of gliadin were deamidated by tTG treatment and assayed for the stimulatory capability on intestinal T cells. T-cell activation was determined by the measurements of IFN-γ production. All gliadin digests were assayed at 50 and 100 μg/ml. The data are mean responses of T-cell lines from 5 different CD patients. Unpaired Student T test was used to assess the statistical significance. *=p<0.05

FIG. 8: SDS-PAGE of the enzyme preparation comprising recombinant E40. Numbered bands were cut and analyzed by proteomic analysis.

FIG. 9: A: SDS-PAGE showing degradation of peanut proteins by recombinant E40; B: HPLC analysis of peanut proteins incubated with recombinant E40 (1:20 enzyme:substrate, e:s) at different incubation times, up to 120 min; for each time point the HPLC of untreated samples is shown in the upper panel, while the lower panel shows the E40 treated samples.

FIG. 10: E40 production in S. lividans in presence/absence of sucrose. Proteolytic activity assayed in supernatant of S. lividans TK24/p1J86/E40 cultures, grown in medium P without Sucrose (♦, Suc0), or with 17% (▪, Suc17) or 34% Sucrose (▴, Suc34). Samples are taken at different fermentation times: A: 120 h; B: 144 h; C: 168 h. Activity is shown as relative fluorescent units (rfu, Y axis) produced during time (minutes, X axis).

FIG. 11: E40 production in Pichia pastoris. Proteolytic activity assayed in supernatant of recombinant P.pastoris cultures, after 50 or 96 hours from E40 expression induction Activity is shown as increase of absorbance (Y axis; milli-absorbance units, mau) during time (X axis; minutes). A and C: samples from cultures grown at pH 6 without or with 34% sucrose, respectively; B and D: samples from cultures grown at pH 7 without or with 34% sucrose, respectively.

FIG. 12: E40 production in Escherichia coli. Proteolytic activity assayed in crude extract obtained from E. coli cells transformed to express E40. Proteolytic activity in the supernatant of S. lividans TK24/p1J86/E40 cultures, grown according to the invention, is reported in the graph for comparison. Activity is shown as relative fluorescent units (rfu, Y axis) produced during time (minutes, X axis).

### DETAILED DESCRIPTION OF THE INVENTION

The present invention is directed to a method for producing an enzyme preparation comprising the mature form of at least one recombinant Actinoallomurus endopeptidase with glutenase activity, comprising in series: culturing a recombinant Streptomyces lividans host cell, preferably of the TK24 strain, in a culture medium under fermentation conditions, said recombinant host cell comprising a recombinant expression vector for heterologous expression of the at least one recombinant Actinoallomurus endopeptidase, the recombinant expression vector comprising a polynucleotide encoding for said at least one recombinant Actinoallomurus endopeptidase, operably linked to regulatory sequences capable of directing the expression of said at least one recombinant Actinoallomurus endopeptidase in the recombinant host cell; recovering the supernatant of the culture medium and purifying from said supernatant an enzyme preparation comprising the mature form of the at least one recombinant Actinoallomurus endopeptidase.

The at least one recombinant Actinoallomurus endopeptidase in the enzyme preparation obtained by the method of the invention is preferably selected from the group consisting of: endopeptidase 40 (E40) of sequence comprising SEQ ID NO: 1; a biologically active fragment of E40; a naturally occurring allelic variant of E40; and an endopeptidase of sequence having at least 60%, 70%, 80%, 90% or 95% of identity to SEQ ID NO: 1.

The term “enzyme (or enzymatic) preparation” is used herein to identify the product obtained at the end of the method of the invention, comprising (enriched in) at least one recombinant Actinoallomurus endopeptidase having glutenase activity; said product can be a composition further including other components.

The term “biologically active fragment” refers to portions of the endopeptidases of the invention, which maintain specific glutenase activity.

A “biologically-active fragment” of an endopeptidase according to the invention can be identified for instance by: isolating a polynucleotide encoding for a fragment of an endopeptidases of sequence SEQ ID NO: 3 or 4 (e.g. a polynucleotidic portion of the polynucleotides of sequence SEQ ID NOs: 5 or 6), expressing the encoded endopeptidase fragment (for example, by recombinant expression in vitro), and verifying by a suitable assay if said endopeptidase fragment has the same glutenase activity of the endopeptidases; a suitable assay is for instance any of the enzyme activity assays disclosed in the present examples.

The method of the invention also encompasses obtaining the enzyme preparation comprising the at least one recombinant Actinoallomurus endopeptidase(s) with glutenase activity by introducing in a S. lividans host cell a recombinant expression vector comprising a polynucleotide having sequence that differs from the nucleic acid sequences SEQ ID NOs: 5 or 6 due to degeneracy of the genetic code and thus encodes the same endopeptidases encoded by a polynucleotide of sequence SEQ ID NOs: 5 or 6.

The endopeptidases obtained by the method of the invention can have sequence that comprises changes in the aminoacidic residues that are not essential for the biological activity of the endopeptidase. The “biological activity” is in this context the natural or normal function of the native Actinoallomurus endopeptidases of sequence SEQ ID NO: 3, for example, it is the ability to degrade gluten proteins.

The method of the invention encompasses obtaining an enzyme preparation comprising at least one recombinant endopeptidases of sequence having at least 60%, 70%, 80%, 90% or 95% of identity to SEQ ID NO: 1. The terms “identity” and “homology” when referred to a nucleotide or aminoacidic sequence are herein used interchangeably and refer to the degree to which two polynucleotide or polypeptide sequences are identical or homologous on a residue-by-residue basis over a particular region of comparison. The alignment and the percent identity or homology can be determined using any suitable software program known in the art, for example those described in Current Protocols in Molecular Biology (Ausubel F. M. et al., “Commercially Available Software”, Current Protocols in Molecular, 1987, Supplement 30, Section 7.7.18, Table 7.7.1). Preferred programs include the GCG Pileup program, FASTA (Pearson R. and Lipman D. J. “Improved Tools for Biological Sequence Analysis” Proc. Natl., Acad. Sci. USA, 1988, 85, 2444-2448), and BLAST (Altschul S. F., Gish W., Miller W., Myers E. W., Lipman D. J. “Basic local alignment search tool” J. Mol. Biol., 1990, 215, 403-410).

The term “allelic variant” denotes any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered aminoacidic sequence. The term allelic variant refers also to a protein encoded by an allelic variant of a gene.

The at least one endopeptidase of the enzyme preparation obtained by the method of the invention can also be operatively-fused to another polypeptide, for instance a tag; preferably the at least one endopeptidase of the enzyme preparation obtained by the method of the invention is a tagged endopeptidase, more preferably a histidine-tagged endopeptidase, most preferably tagged at the C-terminal of the protein, such as the endopeptidase of sequence comprising or consisting of SEQ ID NO: 2.

It is noted that the at least one Actinoallomurus endopeptidase of the enzyme preparation obtained by the method of the invention is in mature form, since the Streptomyces host cell is capable of processing the expressed endopeptidase and of secreting it in the culture medium in mature form. For instance, native E40 has sequence SEQ ID NO: 3 and its mature form thereof has sequence SEQ ID NO: 1. As recombinant endopeptidase, produced in S. lividans host cell according to the method of the invention, E40 is secreted in the culture medium as mature E40 of sequence comprising or consisting in SEQ ID NO: 1, despite the whole coding sequence (SEQ ID NO: 3) was introduced into the host cell.

The enzyme preparation of the invention is preferably enriched in said at least one recombinant Actinoallomurus endopeptidase(s); more preferably the enzyme preparation does not comprise endopeptidases other than the at least one recombinant Actinoallomurus endopeptidase(s); for instance, the enzyme preparation preferably does not comprise endopeptidases of S. lividans origin.

The S. lividans host cell is cultured under fermentation conditions in a suitable culture medium. For “fermentation conditions” it is meant conditions of cultivation of the host cell strain (medium composition, stirring parameters, aeration and temperature) suitable for the strain to grow and to produce the compound of interest (the recombinant endopeptidase). In particular, the culture medium of the fermentation conditions of the method of the invention comprises suitable nutrients of no-animal origin, carbon and nitrogen sources and inorganic salts, and it is characterized in that it comprises at least 30% (wt/vol) sucrose (i.e. at least 30 g of sucrose each 100 ml of medium), preferably about 34% sucrose (wt/vol). Fermentation conditions, according to the present invention, thus comprise culturing the recombinant host cell in a suitable culture medium comprising at least 30% (wt/vol) sucrose; fermentation of the recombinant host cells is carried on at a temperature between 25° C. and 30° C., preferably between 28° C. and 30° C., more preferably of about 30° C.; fermentation is preferably carried on until the pH of the culture medium is greater than 7 and/or until the glucose in the medium is totally consumed and/or until the recombinant protein secreted in the medium has an activity of greater than 8 au/min per mL of supernatant, measured as described herein in example 5. Preferably, the fermentation is carried on for at least 48 hours, preferably for at least 72 hours, before recovering the supernatant of the culture medium for successive purification.

Preferably, the culture medium further comprises yeast extract and soy peptone. A particularly preferred culture medium used in the method of the invention is Medium P comprising Sucrose (about 340 g/L), Glucose (about 20 g/L), Yeast extract (about 3 g/L), Soy peptone (about 5 g/L), Malt extract (about 3 g/L) and having a pH of about 6.7.

Preferably, the method of the invention comprises a first step of culturing a spore suspension of recombinant S. lividans host cells in a first medium comprising glucose, yeast extract and soy peptone at about 30° C. for 3-7 days, preferably for about 4 days, previous to the step of culturing the recombinant host cells under fermentation conditions. More preferably said first medium is Medium V comprising glucose (about 20 g/L), Yeast extract (about 5 g/L), soy peptone (about 10 g/L), NaCl (about 1 g/L) and having pH of about 6.7.

Preferably, suitable amount of an antibiotic is added to the culture media for selection of recombinant host cells, preferably Apramycin, more preferably at 50 mg/L; optionally said antibiotic is added only to the medium of the first step of culturing the host cells and not to the culture medium of the fermentation step.

The host cells may be cultivated by small-scale or large-scale fermentation in laboratory or industrial fermenters.

The recombinant endopeptidase(s) can be recovered directly from the culture medium, since it is secreted therein. The supernatant of the culture medium containing the recombinant endopeptidase can be recovered by methods known in the art, for instance further to centrifugation of the harvested culture.

Further to recovery of the supernatant, the method of the invention preferably comprises one or more steps of purification of the enzyme preparation from said supernatant. The culture medium can be for example filtered off to separate microbial bodies, and the filtrate then processed for the collection of the recombinant endopeptidase(s) by means of one or more of several procedures, such as: ultrafiltration, concentration under reduced pressure, salting out, precipitation by organic solvent, dialysis, gel filtration, adsorption chromatography, ion-exchange chromatography, electro-focusing, and freeze-drying.

Preferably, the purification step(s) of the method of the invention comprise at least a step of purification of the enzyme preparation from the recovered supernatant by affinity chromatography; more preferably it also comprises a step of ultrafiltration; most preferably the purification of the enzyme preparation from the supernatant of the culture medium comprises in series the steps of: filtering the supernatant, preferably through a paper filter and/or through capsule filters having a nominal pore size between 1.0 μm and 0.3 μm, to obtain a clarified solution comprising the recombinant endopeptidase(s) of interest; concentrating the clarified solution by ultrafiltration; purifying the enzyme preparation by affinity chromatography, preferably by immobilized metal affinity chromatography (IMAC), of the ultrafiltered clarified solution, thus obtaining a final enzyme preparation comprising (enriched in) the recombinant endopeptidase(s) of interest. In preferred embodiments, the purification further comprises: depigmentation of the affinity chromatography eluted fractions, preferably by DEAE anion exchange chromatography, concentration and desalting of the depigmented samples, preferably by ultrafiltration of the same, thus obtaining a final enzyme preparation comprising (enriched in) the recombinant endopeptidase(s) of interest.

Representative examples of a preferred method of production of recombinant endopeptidases according to the invention are provided hereinafter in Examples 2-4 and 15.

Preferably, the recombinant expression vector that is introduced in the host cell in order to express the endopeptidase(s) of interest comprises a polynucleotide that encodes for E40 of sequence SEQ ID NO: 1 or for a derivative thereof, preferably the polynucleotide being of sequence SEQ ID NO: 5 or SEQ ID NO: 6, encoding respectively for E40 having sequence consisting of SEQ ID NO: 3 or for a C-terminal histidine tagged E40 having sequence consisting of SEQ ID NO: 4; the polynucleotide may also be of sequence having at least 60%, 70%, 80%, 90% or 95% of identity to SEQ ID NO: 5 or SEQ ID NO:6. The recombinant S. lividans host cell comprising said expression vector is capable of producing the endopeptidase(s) of interest and secreting the mature form thereof in the culture medium. For instance, the mature form of E40 of sequence SEQ ID NO: 3 is the endopeptidase of sequence SEQ ID NO: 1, and the mature form of the histidine-tagged E40 of sequence SEQ ID NO: 4 is the endopeptidase of sequence SEQ ID NO: 2.

The production of an enzyme preparation comprising a histidine tagged endopeptidase, such as a histidine-tagged E40, by the method of the invention is of particular interest. Therefore, in a preferred embodiment, the invention is directed to a method according to claim 1, wherein the recombinant expression vector comprises a polynucleotide encoding for a histidine tagged E40 of sequence comprising SEQ ID NO: 2, preferably wherein the polynucleotide is of sequence SEQ ID NO: 6 and the enzyme preparation purified from the supernatant of the culture medium comprises the mature form of the histidine tagged E40 of sequence SEQ ID NO: 4.

Preferably, the recombinant expression vector used in the method of the invention is recombinant pIJ86 expression vector. The present invention is therefore also directed to a recombinant pIJ86 expression vector, comprising a polypeptide having sequence comprising SEQ ID NO: 5 or SEQ ID NO:6, preferably to the recombinant pIJ86 expression vector having sequence SEQ ID NO: 8.

The present invention is further directed to a recombinant S. lividans host cell, preferably of strain TK24, comprising said recombinant expression vector; more preferably said host cell is of strain DSM 33207, obtained as described herein according to the invention, and deposited on July 17, 2019 with the Leibniz Institute DSMZ-German Collection of Microorganisms and Cell Cultures GmbH, InhoffenstraSe 7B 38124 Braunschweig—GERMANY, under the provision of the Budapest Treaty.

The present invention is also directed to the enzyme preparation that is obtained by the method of the invention, comprising the mature form of the at least one recombinant Actinoallomurus endopeptidase having glutenase activity.

In a preferred embodiment, the enzyme preparation is in powder form.

In a preferred embodiment, the recombinant Actinoallomurus endopeptidase(s) comprised in the enzyme preparation is isolated from the supernatant of the culture medium or from the enzyme preparation obtained after purification of the culture medium. The present invention is then directed also to an isolated recombinant Actinoallomurus endopeptidase, obtained from the supernatant recovered from the culture medium of the recombinant S. lividans host cell in the method of the invention or from the final enzyme preparation obtained after purification step(s) according to the method of the invention. Preferably the isolated recombinant Actinoallomurus is selected from the group consisting of: recombinant endopeptidase 40 (E40) of sequence comprising SEQ ID NO: 1; a biologically active fragment of E40; a naturally occurring allelic variant of E40; and an endopeptidase of sequence having at least 60%, 70%, 80%, 90% or 95% of identity to SEQ ID NO: 1. More preferably, it is an endopeptidase of sequence consisting of SEQ ID NO: 1 or 2, or of sequence having at least 60%, 70%, 80%, 90% or 95% of identity to SEQ ID NO: 1 or 2.

Optionally, the enzyme preparation obtained by the method of the invention can be administered orally directly after purification to a subject in need thereof. Preferably, the enzyme preparation or the isolated recombinant Actinoallomurus endopeptidase is formulated in a pharmaceutical formulation suitable for human use.

The present invention is therefore also directed to a pharmaceutical formulation comprising as the active proteolytic ingredient the enzyme preparation or the isolated recombinant Actinoallomurus endopeptidase(s) obtained by the methods of the invention.

A preferred pharmaceutical formulation is compatible with its intended route of administration, which according to the instant invention is preferably the oral administration. The pharmaceutical formulation of the invention is thus preferably an oral pharmaceutical formulation. Pharmaceutical formulations according to the invention can be prepared with the appropriate ingredients to generate a preparation in liquid form, for example in the form of a solution, emulsion, or in solid form, such as tablets, capsules, semisolid or powder. The pharmaceutical formulation of the enzyme preparation can be administered in a variety of ways including those particularly suitable for admixing with foodstuff. The recombinant Actinoallomurus endopeptidase(s) with glutenase activity present in the pharmaceutical formulation can be active prior to or during ingestion, and may be treated, for example, by a suitable encapsulation, to control the timing of activity.

To prepare an appropriate pharmaceutical formulation according to the present invention any method for the stabilization of chemical or biological material known in the art, comprising those based on irradiation or temperature modulation or their combinations, can be used.

The pharmaceutical formulations of the invention are more preferably formulated so as to release their active ingredients in the gastric fluid. This type of formulations will provide optimum activity in the right place, for example by releasing the enzyme preparation of the invention in the stomach of a subject in need thereof.

The present invention also includes a food or a food supplement which comprises as active proteolytic ingredient the enzyme preparation or the isolated recombinant Actinoallomurus endopeptidase obtained by the methods of the present invention.

The enzyme preparation or the isolated recombinant endopeptidase, obtained by the method of the invention, can also be formulated as food supplement, prepared, supplied and dispensed as described in other prior documents regarding the field of this invention (WO2011/077359, WO 2003/068170, WO2005107786). As an example, the food supplement of the invention may be a granulated enzyme-coated or -uncoated product which may readily be mixed with food components; alternatively, food supplements of the invention can form a component of a pre-mix; alternatively, the food supplements of the invention may be a stabilized liquid, an aqueous or oil-based slurry. The pharmaceutical composition or the food supplement of the invention can be provided prior to meals, immediately before meals, with meals or immediately after meals, so that the endoproteases of the enzyme composition of this invention are released or activated in the upper gastrointestinal lumen where the endoproteases can complement gastric and pancreatic enzymes to detoxify ingested gluten and prevent harmful peptides to pass the enterocytes layer.

The enzyme preparation can also be formulated as a food; in a preferred embodiment said food is a flour that has been put in contact with said enzyme preparation or said isolated recombinant Actinoallomurus endopeptidase capable of degrading gluten.

Alternatively, the enzyme preparation and the isolated recombinant Actinoallomurus endopeptidases of the invention may also be used to produce protein hydrolysates for food and/or drinks.

Preferably, the enzyme preparation, the isolated recombinant Actinoallomurus endopeptidase, the pharmaceutical formulations, the food or food supplement of the present invention are used in the treatment and/or prevention of celiac disease or of a disorder associated to celiac disease or of non-celiac gluten sensitivity, preferably of any disorder associated with intolerance to gliadin peptide of sequence SEQ ID NO:6. Disorders associated to celiac disease include: celiac sprue, dermatitis herpetiformis, celiac disease mucosal damage, and diseases consequent to celiac disease mucosal damage, such as iron-deficient anemia, osteoporosis, type-1 diabetes, autoimmune thyroiditis and enteropathy-associated T-cell lymphomas.

Moreover, the Actinoallomurus endopeptidase with glutenase activity are also capable of preventing and/or treating allergy and/or intolerance to nuts and/or peanuts. The present invention is therefore further directed to said use.

Disorders selected from the group consisting of: celiac disease, a disorder associated to celiac disease, non-celiac gluten sensitivity and allergy or intolerance to nuts and/or peanuts, can thus be prevented and/or treated by administration to a subject in need thereof of an effective amount of the enzyme preparation, or of the isolated recombinant Actinoallomurus endopeptidase, optionally formulated as pharmaceutical formulation, food or food supplement of the invention, more preferably by oral administration.

The present invention is thus further directed to a method of treating and/or preventing a disorder selected from the group consisting of: celiac disease, a disorder associated to celiac disease, non-celiac gluten sensitivity, and allergy or intolerance to nuts and/or peanuts, by administering to a subject in need thereof an effective amount of the claimed enzyme preparation or of the claimed isolated recombinant Actinoallomurus endopeptidase, or of the claimed pharmaceutical formulation, food or food supplement. Depending on the patient and condition being treated and on the administration route, the active amount may be a dosage corresponding to 0.01 mg to 0.5 mg of recombinant endopeptidase for each kg of body weight per day, e. g. about 20 mg/day for an average person. A typical dose of glutenase in patients will be at least about 1 mg/adult, more usually at least about 10 mg; and preferably at least about 50 mg; usually not more than about 5 g, more usually not more than about 1 g, and preferably not more than about 500 mg. Dosages will be appropriately adjusted for pediatric formulation. In children the effective dose may be lower, for example at least about 0.1 mg, or 0.5 mg. Those of skill will readily appreciate that dose levels can vary as a function of the specific enzyme, the severity of the symptoms and the susceptibility of the subject to side effects.

### EXAMPLES

Reagents used in the Examples

Actinomycete strain Actinoallomurus A8 was from the collection of “Fondazione Istituto Insubrico di Ricerca per la Vita”. The N-Succinyl-Ala-Ala-Pro-Phe p-nitroanilide (suc-Ala-Ala-Pro-Phe-pNA) and N-Succinyl-Ala-Ala-Pro-Phe-7-aminomethyl coumarine (suc-Ala-Ala-Pro-Phe-AMC) were from Bachem (Bubendorf, Switzerland); pepsin, trypsin, nalidixic acid, and apramycin were from Sigma-Aldrich (Milan, Italy); mannitol was from Carlo Erba (Cornaredo, Italy); soya flour was from Cargill (Padova, Italy); agar and soy peptone were from Conda (Madrid, Spain); sucrose and glucose were from VWR (Leuven, Belgium); yeast and malt extracts were from BD (Franklin Lakes, N.J., USA) and Costantino (Favria, TO, Italy) respectively; the 33-mer a-gliadin peptide was synthesized by Biotem (Apprieu, France); strain S. lividans TK24 and expression vector pIJ86 were from the John Innes Center (Norwich, UK); Saccharomyces cerevisiae type strain X4004-3A (Accession no. KR348914) was supplied from Prof. L. Pollegioni.

Example 1—Native E40 Activity

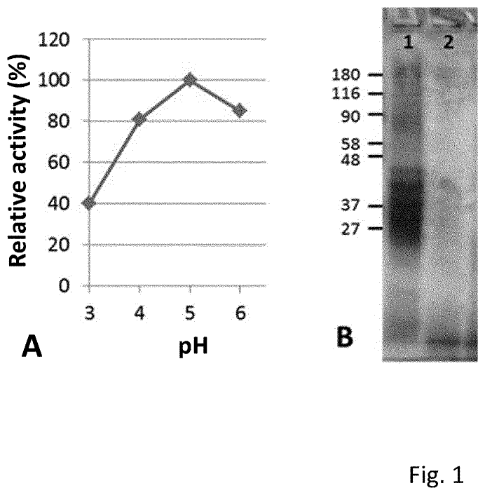

Native E40 of sequence SEQ ID NO: 3 was purified from the supernatant fraction of an Actinoallomurus A8 culture isolated from soil (native E40) as described in WO2013083338. Native E40 shows marked glutenase activity at environmental condition of pH 3-6, with optimum at pH 5 (FIG. 1, panel A). At these conditions, gliadin proteins are completely digested (FIG. 1, panel B).

Example 2-E40 coding gene cloning in pIJ86 expression vector and insertion in S. lividans TK24 host cell.

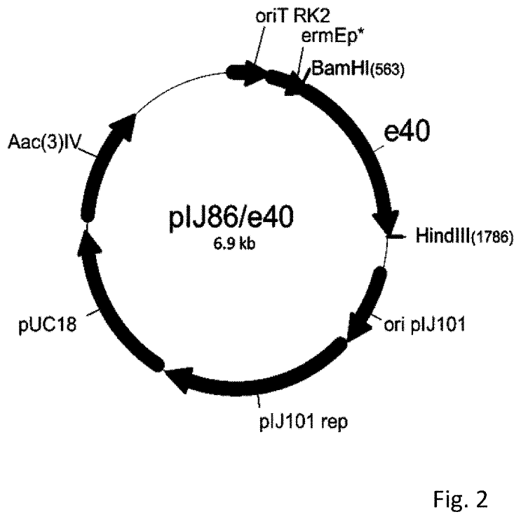

A polynucleotide of sequence SEQ ID NO: 6, encoding for a histidine-tagged E40 was amplified from Actinoallomurus A8 genomic DNA using Phusion High Fidelity polymerase (Thermo Scientific, Rodano, MI, Italy) and 0.5 μM of oligonucleotide primers (SEQ ID NOs: 9 and 10), that introduced BclI and HindIII restriction sites at the 5′ and 3′ ends of the polynucleotide sequence and one glycine followed by six histidine residues at the C-terminus of the endopeptidase. PCR Cycling conditions were: 3 minutes at 98° C., followed by 10 cycles of 10 s at 98° C., 20 s at 67° C. and 50 s at 72° C., 20 cycles of 10 s at 98° C., 70 s at 72° C., with a final extension at 72° C. for 5 minutes. The PCR products (SEQ ID NO: 11) were purified from an agarose gel, digested with HindIII and BclI and ligated in an empty pIJ86 vector (SEQ ID NO: 7) digested with HindIII and BamHI to produce the recombinant vector pIJ86/e40 of sequence SEQ ID NO: 8 (FIG. 2). The absence of PCR-generated mutations in the recombinant vector was assessed by DNA sequencing. The pIJ86/e40 vector, bearing e40 of sequence SEQ ID NO: 6 under the control of the strong constitutive ermE promoter, was used to transform E. coli ET12567/pUZ8002. Conjugations were performed according to Flett et al ¹⁴ using E. coli ET12567 as donor and S. lividans TK24¹⁷ as recipient, thus obtaining a recombinant S. lividans TK24 host cell bearing the E40 coding gene (T24/pIJ86/e40 host cell). The mating mixture was spread onto Mannitol Soya flour agar plates (Mannitol 8 g/L, Soya flour 8 g/L, Agar 8 g/L, MS) containing 10 mM MgCl₂ and incubated at 28° C. After 16-20 h, 1 ml distilled water containing 50 μg/ml nalidixic acid and 30μg/ml Apramycin was added to the surface of each plate and spread using a glass rod and further incubated at 28° C. until exconjugant colonies appeared. The exconjugants were repeatedly plated on to MS agar containing nalidixic acid and apramycin; a final spore suspension, prepared according to Kieser et al¹⁵, was stored at −80° C. in 20% glycerol.

### Example 3—Expression of E40 by recombinant S. lividans TK24

The recombinant S. lividans TK24/p1J86/e40 host cells of Example 2 were inoculated in Erlenmeyer flasks (50 ml) containing 20 ml of Medium V (as defined above) added with Apramycin 50 mg/L and incubated on a rotatory shaker at 200 rpm at 30° C. After 5 days of growth, the culture was inoculated in one Erlenmeyer flask (500 ml) containing 100 ml of the Medium P (as defined above) added with Apramycin 50 mg/L and incubated in the same conditions for 8 days. Alternatively, for a 15 L scale fermentation, the 5-days culture grown in Medium V was first inoculated in three 500 ml-Erlenmeyer flasks containing 100 ml of the Medium V, and incubated in the same conditions; then, after 72 hours, the flask cultures were harvested, pooled and used to inoculate a fermenter (Biostat Cplus, Sartorius Stedim, Goettingen, Germany) containing 15 liters of Medium P added with Apramycin 50 mg/L. Fermentation was run at 30° C. under stirring conditions of 450 rpm and recombinant E40 production was monitored over the time by measuring the enzymatic activity in the culture supernatant, obtained after centrifugation at 11000 g for 6 min. Fermentation was stopped after 94 hours, when enzymatic activity reached 1430 U per ml of supernatant (see example 5 for the enzymatic activity assay used herein).

Example 4—Purification of the Enzyme Preparation from the Supernatant of the S. lividans TK24/p1J86/e40 Culture Medium

The harvested S. lividans TK24/p1J86/e40 culture of example 3 was centrifuged for 90 min at 4120 g and the recovered supernatant was collected and filtered on Rapida A paper (Enrico Bruno, Turin, Italy) and further clarified two times onto polyethersulfone opticap capsules (nominal pore sizes 1.0 μm and 0.5 μm) (Merck, Vimodrone, Italy). Next, the clarified solution was concentrated 10 times by ultrafiltration system (TFF1) using Pellicon 3 Ultracel TFF cellulose cassettes (10 kD nominal MW cutoff) and added to 0.5 M NaCl and 50 mM Na₂HPO₄ pH 7.2. Enzyme was purified by Immobilized Metal Affinity Chromatography (IMAC) by using Ni Sepharose® 6 Fast Flow resin (GE Healthcare, Milan, Italy). IMAC column was equilibrated with 5 volumes of phosphate buffer (pH 8.0), elution was carried out by 5 volumes of 250 mM imidazole, 50 mM phosphate buffer (pH 8.0), eluted fractions (330 ml) were adjusted to pH 6.3 with formic acid and depigmented by DEAE anion exchange chromatography (GE Healthcare). Depigmented samples were concentrated and desalted by ultrafiltration system (TFF2, Pellicon, 10 kD MW nominal cut-off). Before freeze-drying, sample was added with mannitol and trehalose (15 and 5 mg/ml, respectively), to improve the crystallization process¹⁶.

The protein content of purified sample was 6% (measured by BCA assay) with 41000 U/mg protein, equivalent to about 8 mg protein/ml of culture supernatant.

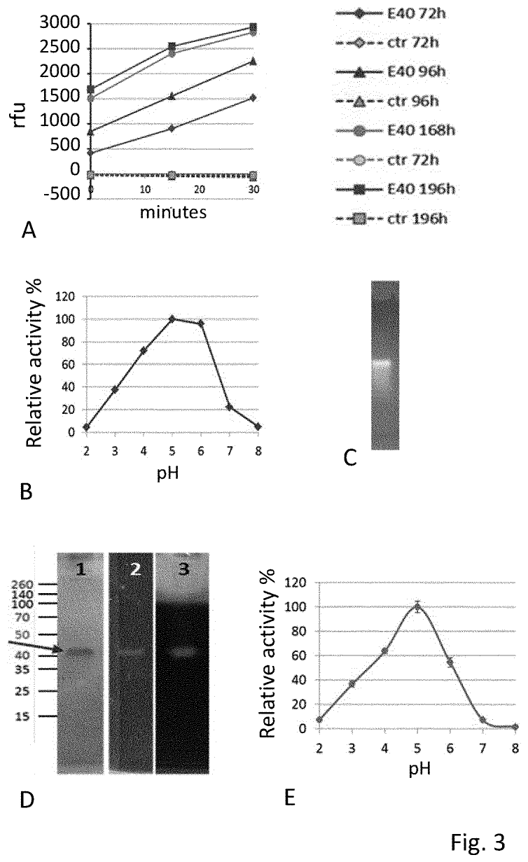

The efficiency of the purification process was demonstrated by SDS-PAGE, where recombinant E40 migrated in the main band at ˜40 kDa (FIG. 3, panel D, lane 1, arrow). The N-terminal sequence analysis of the 40 kDa SDS-PAGE band confirmed the predicted mature form of recombinant E40.

Example 5-Enzymatic Activity of the Enzyme Preparation.

Enzymatic activity assays were performed in 96-well microtiter plates (transparent, flat-bottom) using the Infinite 200 PRO plate reader (Tecan, Mannedorf, CH). Twenty microliters of samples comprising E40 in a concentration range 10-50 nM, were pre-incubated for 5 min at 37° C., and then added to 180 μl of 220 μM suc-Ala-Ala-Pro-Phe-pNA in citrate phosphate buffer (0.1M citric acid, 0.2 M disodium hydrogen phosphate, pH 5). Samples were incubated for 20 minutes at 37° C. The pNA was detected at 410 nm, reading at interval of 5 min.

Enzyme activity is determined by the linear increase of absorbance during time: 1 Activity Unit (AU) is defined as the amount of enzyme able to release 1 arbitrary absorbance unit (aau) in 1 minute and measured as “arbitrary absorbance units produced per minute” (aau/m):

$$1 AU = aau / m = \frac{A b {s}^{T 2} - A b {s}^{T 1}}{T 2 - T 1}$$

It is usually calculated in the 5-15′ interval (T1=5 and T2=15 minutes, respectively).

To correctly evaluate E40 production, any sample must be diluted to give a linear increase in absorbance with time; dilution 1:40 is used for supernatant samples from the currently used E40 producer. E40 production is expressed as AU/mL of supernatant.

The same E40 quantification method is used for samples derived by all the steps of the down-stream process, and expressed as AU/mL or AU/mg for liquid or solid samples, respectively.

1 Unit (U) is defined as the amount of enzyme that released 1 μmole of pNA per minute. In our assay conditions: au per pmole of pNA=0.006; 1 U=AU/0.006. Sample activity was expressed as enzyme Units per ml (in case of supernatant samples) or per mg of solid material (in case of final powder enzyme preparation containing recombinant E40).

The pH optimum was determined using the same substrate which was prepared in 0.2 M ammonium citrate for pH 2 or citrate phosphate buffer at pH range 3-8, respectively.

For the zymography analysis, E40 containing samples were run by non-reducing SDS-PAGE (12% polyacrylamide) at 100 V using a Tetra-cell Mini-PROTEAN system (Bio-Rad, Milan, Italy). Gel was washed twice with citrate-phosphate/phosphate buffer (pH 5.0), and then incubated with the same buffer including 100 μM of suc-Ala-Ala-Pro-Phe-AMC. The activity of E40 was visualized in the gel exposed by UV-light.

The proteolytic activity of E40 was also evaluated by using gelatin as substrate, after electrophoretic running, gel was washed with citrate phosphate buffer pH 5.0 and overlapped onto a zymogram 10% gelatin gel (Bio-rad) equilibrated with the same buffer for 10 minutes. The two overlapped gels were incubated at 37° C. for 2 hours, then the gelatin gel was stained with Coomassie Brilliant R250. Gelatin digestion was visualized after gel destaining as clear lysis bands, due to proteolytic action of proteases diffused from the PA-gel to the zymogram one.

The monitoring of the enzymatic activity in the supernatant of the cultures grown in flasks in example 3 demonstrated a stable and continuous production of recombinant E40, up to 8 days of fermentation (FIG. 3, panel A). As well as native E40, recombinant E40 was active in the pH range 3-6, with an optimum at pH 5 (FIG. 3, panel B). The zymographic analysis, carried out at pH 5, demonstrated that recombinant E40 was the only active protein in culture supernatants (FIG. 3, panel C).

Recombinant E40 is also the only protein in the purified enzyme preparation obtained in example 4 that is active toward the chromogenic substrate suc-Ala-Ala-Pro-Phe-AMC (FIG. 3, panel D, lane 2) and also against a generic protein substrate like gelatin (FIG. 3, panel D, lane 3), thus excluding the presence of any endoprotease other than recombinant E40 in the final purified enzyme preparation. Activity of purified enzyme preparation comprising recombinant E40 enzyme preparation at different pH values was evaluated with the substrate suc-Ala-Ala-Pro-Phe-pNA, pH range 2 to 8 (FIG. 3, panel E). It resulted to be maximally active at pH 5 and significant residual activity (˜40%) was maintained at pH 3. These results predict optimal action in a post-prandial gastric environment, when pH is higher than 3 for over an hour^{8,9}. Interestingly, recombinant E40 remains significantly active (approximately 50%) at pH 6, suggesting also a prolonged action into the post-prandial duodenal compartment where pH is below or equal to 6 for long time, after the meal⁹. Overall, the results support a perfect similarity between the native and recombinant form of E40. The purified enzyme preparation comprising the recombinant E40, obtained in example 4 by a method according to the invention, has in fact chemical/physical properties and bioactivity comparable to the native E40 isolated from Actinoallomurus strain.

Example 6-Recombinant E40 is Resistant to Digestive Proteases.

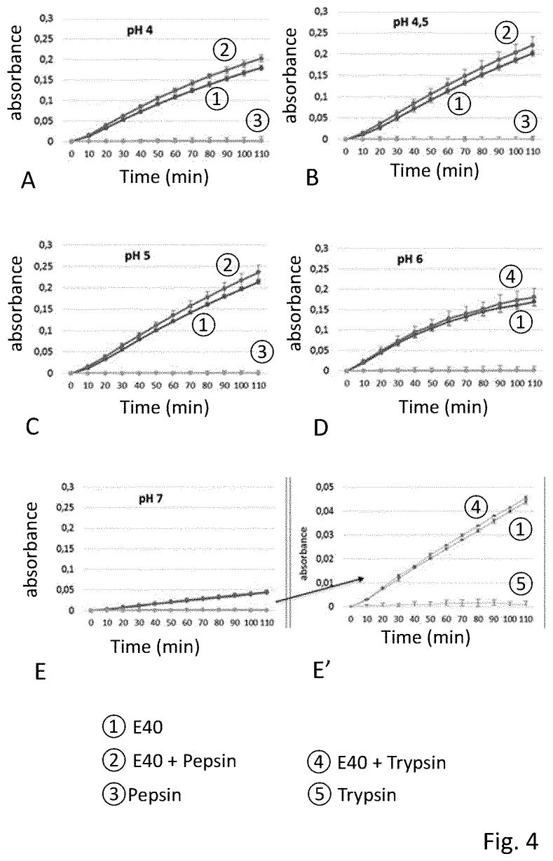

The resistance of recombinant E40, obtained by the method of the invention, to the proteolytic activity of gastric (pepsin) and duodenal (trypsin) digestive proteases was further evaluated (FIG. 4), using suc-Ala-Ala-Pro-Phe-pNA as substrate. Aqueous solutions of E40 enzyme, alone or in combination with pepsin and/or trypsin (0.2 mg/ml E40, 0.1 mg/ml Pepsin or Trypsin), were diluted in citrate-phosphate buffer containing suc-Ala-Ala-Pro-Phe-pNA at pH 4, 4.5 and 5 for pepsin, and at pH 6 and 7 for trypsin. Final concentrations in reaction mix were: 200 μM substrate, 1-4 nM E40 for incubations at pH 4 to 5, or 4 nM and 7 nM for incubations at pH 6 or 7, respectively (E40 concentration was adjusted according to optimum pH). The digestive protease was maintained in the ratio 1:2 (w/w) versus E40.

Reactions (200 μl volume in 96-wells flat bottom ptiter plates) were protracted for 110 minutes at 37° C. Absorbance was monitored every 10 min for determining the enzymatic activity. Pepsin and trypsin without E40 were processed in the same way and tested in the same condition as reference control. Each analysis was carried out in duplicate.

Noteworthy, neither pepsin (FIG. 4, panels A-C) nor trypsin (FIG. 4, panels D-E, E′) hydrolyze the chromogenic substrate. Notably, E40 cleaving activity was unaffected either by pepsin/trypsin addition or by acidic pH. By contrast, a slightly enhanced E40 activity was observed in the presence of pepsin (FIG. 4, panels A-C), maybe due to unmasking of E40 catalytic site by pepsin. Overall, these findings confirm E40 resistance to the main human digestive proteases, at physiological conditions.

Example 7-Digestion of the Immunodominant 33-Mer Gliadin Peptides by E40.

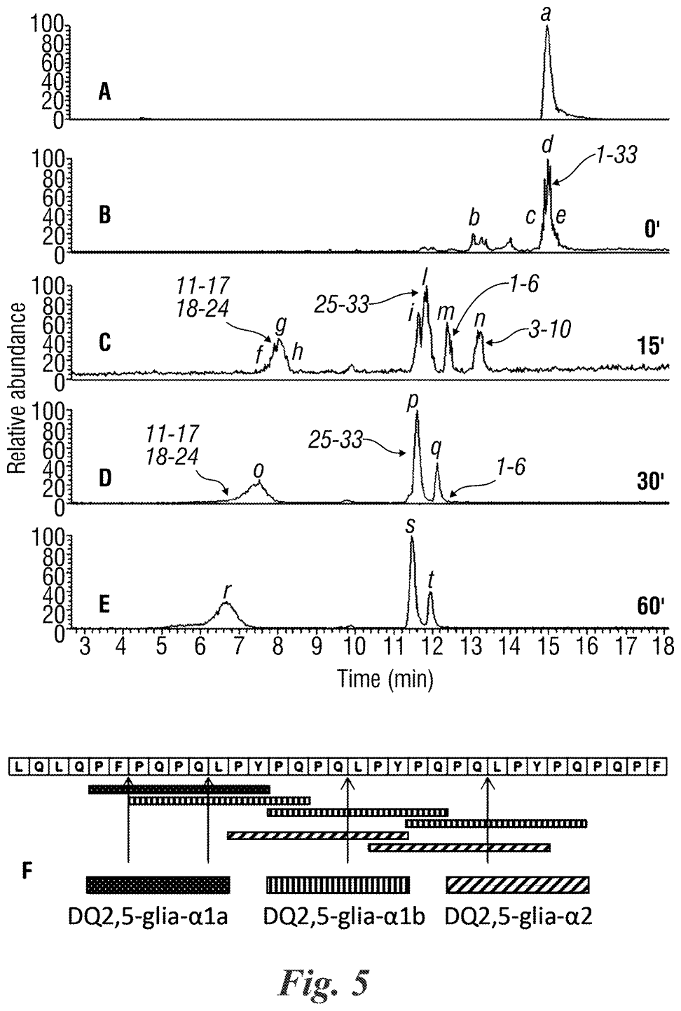

The 33-mer gliadin peptide degradation was monitored by LC-MS/MS, before (FIG. 5, panel A) and after (FIG. 5, panels B-E) digestion by recombinant E40. Digestion was performed in U-bottom 96-wells microtiter plate; 33-mer was diluted in citrate phosphate buffer (pH 5) to 5 mg/ml, mixed with the enzyme preparation comprising recombinant E40 diluted in the same buffer (1:48 enzyme vs substrate molar ratio) and incubated at 37° C. At the time points 0, 15, 30 and 60 minutes, aliquots (10 μl) were taken, and reactions were stopped by boiling for 3 minutes before being analyzed by LC-MS/MS.

Samples were analyzed by HPLC system Accela Instrument (Thermo Fisher Scientific, San Jose, Calif.) coupled to both UV detector and LTQ-XL ion trap mass spectrometer (Thermo Fisher Scientific). Notably, a slight hydrolysis of 33-mer was observed as soon as the incubation started (FIG. 5, panel B, minutes); the native 33-mer peptide signal completely disappeared after only 15 minutes (FIG. 5, panel C). More specifically, the MS/MS spectra analysis demonstrated that E40 activity broke down all the 33-mer immunotoxic sequences. E40 cleaving site occurs between F-P and Q-L residues of gliadin, leading to short peptides lacking of T-cell stimulatory capacity (FIG. 5, F)³. This finding was also observed at more diluted E40 concentration (1:96, molar ratio): in that case complete 33-mer degradation took 30 minutes (not shown).

Example 8-Digestion of whole Gliadin by E40.

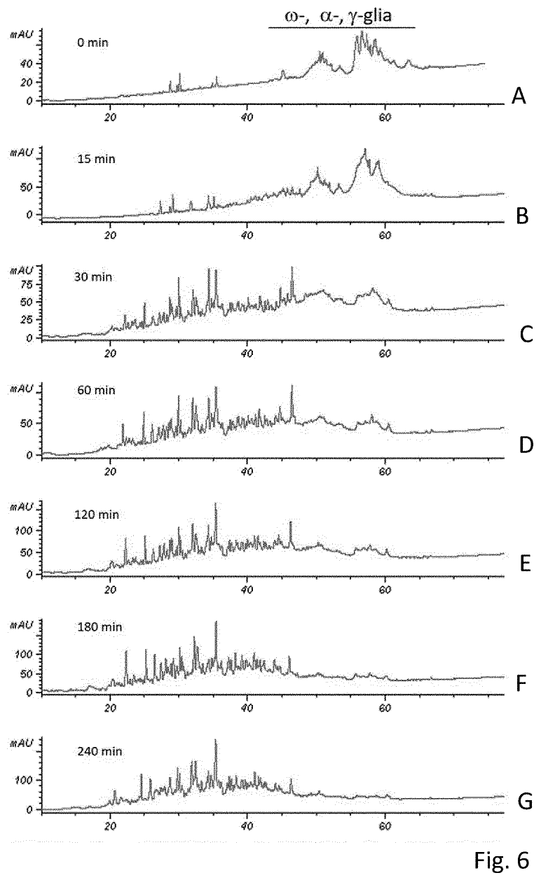

The ability of E40 to hydrolyze harmful peptides in whole gliadin proteins was assessed by HPLC analysis (FIG. 6). Gliadin was extracted from whole flour of Triticum aestivum (Sagittario cultivar) according to Mamone et al⁴. Gliadin (1 mg) was dissolved in 1 ml of 0.1 M ammonium acetate pH 4 and incubated with E40 enzymes (enzyme:substrate, 1:20), at 37° C. for different time points (0, 15, 30, 60, 120, 240 minutes). The enzymatic hydrolysis was stopped by boiling samples for 5 minutes. Samples were lyophilized and stored at −40° C. until further chemical analysis.

SDS-PAGE was performed on a Tetra-cell Mini-PROTEAN system (Bio-Rad, Milan, Italy). Digested gliadin samples were dissolved in Laemli buffer (0.125 M Tris-HCl pH 6.8, 5% SDS, 20% glycerol, 0.02% bromophenol blue) and loaded onto precast 12% acrylamide gel (Bio-Rad). Electrophoresis was carried out under non-reducing conditions, omitting β-mercaptoethanol in the Laemli buffer. Protein bands were visualized with silver blue (Coomassie Brilliant Blue G-250) and digitalized using a LABScan scanner (Amersham Bioscience/GE Healthcare, Uppsala, Sweden). RP-HPLC was carried out on a RP-HPLC using an HP 1100 Agilent Technology modular system (Palo Alto, Calif., USA). Digested gliadin samples were suspended in 0.1% TFA and separated by C18 column (Aeris PEPTIDE, 3.6 μm, 250×2.10 mm i.d., Phenomenex, Torrance, Calif., USA). Eluent A was 0.1% TFA (v/v) in Milli-Q water, eluent B was 0.1% TFA (v/v) in acetonitrile. The column was equilibrated at 5% B. Peptides were separated applying a linear 5-70% gradient of B over 90 minutes at a 0.2 mL/min flow rate. Chromatographic separation was performed at 37° C., using a thermostatic column holder. The column effluent was monitored at 220 and 280 nm with an UV-Vis detector.

Because of the complex mixture of whole gliadins, digestion with the enzyme preparation comprising recombinant E40 (1:48, molar ratio) was extended up to 240 minutes of incubation (FIG. 6, panel B-G). Undigested gliadin sample was run as reference control (FIG. 6, panel A). As expected, the chromatographic profile was drastically changed, since peaks assigned to α-, β-, and γ-gliadin were markedly reduced after 30 min (FIG. 6, panel C) and disappeared between 60 and 120 min of E40 digestion (FIG. 6, panels D and E). The profiles of digested products at 180 min and 240 min (FIG. 6, panels F and G) were similar, indicating that no further degradation arose.

Example 9-E40 Proteolytic Degradation of Humoral and T-cell Gluten Epitopes.

Next it has been evaluated whether recombinant E40 enzymatic activity efficiently neutralizes: i) the capacity of gluten proteins to bind G12 antibodies, and ii) the stimulation of a T-cell-mediated immune response, measured by IFN-γ release. To this purpose, gliadin proteins were suspended in 1 ml of 0.1M ammonium acetate and digested with E40 alone, or with E40 co-incubated with the gastrointestinal proteases, pepsin and trypsin/chymotrypsin, at different pHs and time points, as detailed in Table 1.

TABLE 1

| Gliadin | | |
|---|---|---|
| digest | Gastric hydrolysis | Duodenal hydrolysis |
| sample | condition | condition |
| A | E40 (1 h) (pH 4) | |
| B | E40 (2 h) (pH 4) | |
| C | E40 + Pepsin (1 h) | |
| | (pH 4) | |
| D | E40 + Pepsin (2 h) | |
| | (pH 4) | |
| E | E40 + Pepsin (2 h) | Trypsin + |
| | (pH 4) | Chymotrypsin (2 h) |
| | | (pH 6) |
| F | E40 + Pepsin (2 h) | Trypsin + |
| | (pH 4) | Chymotrypsin (4 h) |
| | | (pH 6) |
| G | E40 + Pepsin (1 h) | Trypsin + |
| | (pH 4) | Chymotrypsin (2 h) |
| | | (pH 6) |
| H | E40 + Pepsin (1 h) | Trypsin + |
| | (pH 4) | Chymotrypsin (4 h) |
| | | (pH 6) |
| I | Pepsin (2 h) (pH 4) | Trypsin + |
| | | Chymotrypsin (4 h) |
| | | (pH 6) |
| L | Pepsin (2 h) (pH 4) | Trypsin + |
| | | Chymotrypsin (4 h) |
| | | (pH 7) |

All enzymes were added in w/w ratio 1:20 (enzyme:protein) and incubated at 37° C., at indicated pH and incubation time. E40/pepsin/chymotrypsin-digested gliadins were deamidated by tTGase (Sigma Aldrich), as previously described⁴. Briefly, the enzymatic gliadin digests (500 μg/ml) were incubated with 2 U of guinea pig tTG (T-5398, Sigma, St. Louis, Mo., USA) at 37° C. for 2 hours in PBS with 1 mmol/L CaCl2.

To estimate the effect of E40 digestion on the immunological potential of wheat, gluten content of samples A-L was determined by monoclonal antibodies specific for QPQLPY sequence (Elisa-G12)¹⁰.

Compared to untreated gliadin samples (samples I and L), E40 digested sample (samples from A to H) showed a drastic reduction of gluten content, well below 20 ppm (Table 2).

TABLE 2

| Gliadin digest | ppm |
|---|---|
| sample | (ng/mL) |
| A | 1.72 |
| B | 2.26 |
| C | 5.23 |
| D | 5.42 |
| E | 4.09 |
| F | 2.68 |
| G | 1.68 |
| H | 2.05 |
| I | >800 |
| L | >800 |

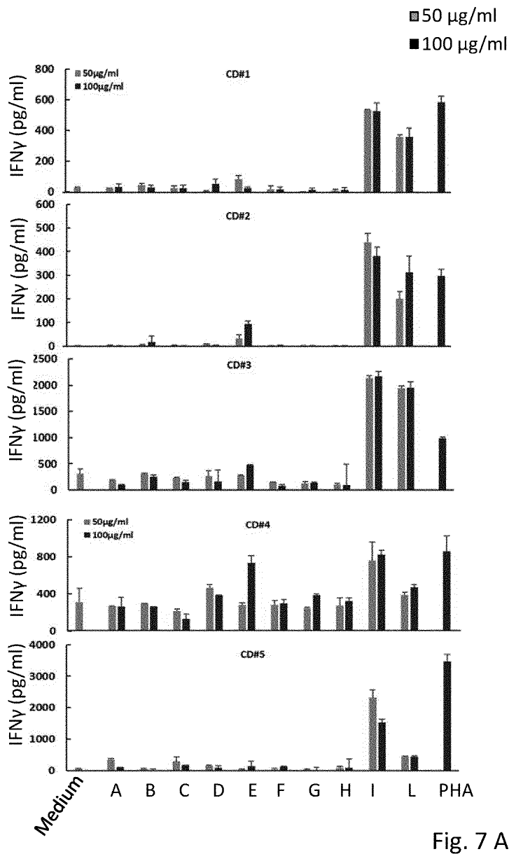

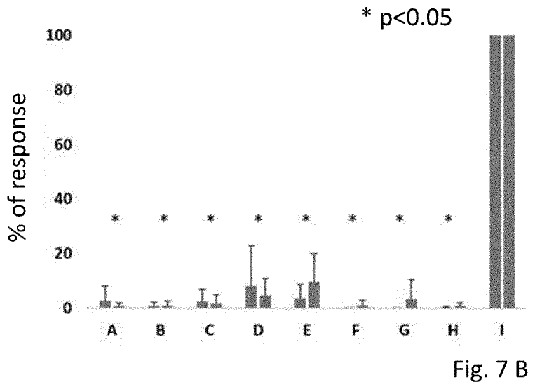

In order to evaluate the capability of gliadin preparations to activate a T-cell response, the different gliadin enzymatic digests (samples A-L in FIGS. 7A and 7B) deamidated by tTG treatment, were assayed on CD4+ T-cell lines previously established from intestinal biopsies of CD patients¹¹ (Table 3).

TABLE 3

| | | | Peptide |
|---|---|---|---|
| Patients | Diagnosis | Age | recognition |
| CD #1 | atrophic | 6 yrs | DQ2, 5-glia-γ1 |
| CD #2 | potential | 2 yrs & 7 m | DQ2, 5-glia-α1, 2; |
| | CD | | ω1, 2 |
| CD #3 | atrophic | 3 yrs & 8 m | DQ2, 5-glia-ω1, 2; |
| | | | γ26mer |
| CD #4 | atrophic | 15 yrs | DQ2, 5-glia-ω1, 2; |
| | | | γ26mer |
| CD #5 | remission | 18 yrs | DQ2, 5-glia-α1, 2 |

Briefly, intestinal mononuclear cells were stimulated with irradiated autologous peripheral blood mononuclear cells (PBMCs) and deamidated pepsin-chymotrypsin digested gliadin extracted from the hexaploid wheat. Growing T cells were kept in culture by repeated stimulation with heterologous irradiated PBMCs and PHA, and IL-2 as a growth factor. The peptide specificity of the TCLs was evaluated by assaying their reactivity toward a panel of immunogenic gluten peptides, and revealed a large repertoire of peptide recognition. In the functional assays, the immune response of TCLs to gliadin enzymatic digests (samples A-L, Table 1 and FIGS. 7A and B) was assayed by detecting IFN-γ production. T cells (3×10⁴) were co-incubated with irradiated autologous EBV-transformed, B-lymphoblastoid cell lines (B-LCLs, 1×10⁵), in 200 μL of complete medium (X-Vivol5 supplemented with 5% human serum and penicillin-streptomycin antibiotics, all reagents supplied by Lonza-BioWhittaker, Verviers, Belgium) in U-bottom 96-well plates. After a 48 hours incubation, cell supernatants (50 μL) were collected for IFN-γ determination. Each antigen preparation was assayed in duplicates and in at least three independent experiments for each T-cell line. IFN-γ was measured by a classic sandwich ELISA using purified and biotin-conjugated anti-IFN-γ Abs purchased from Mabtech (Nacka Strand, Sweden). The sensitivity of the assay was 32 pg/ml.

As expected, all the T cells lines produced high level of IFN-γ when exposed to pepsin-chymotrypsin/trypsin digested gliadin (samples I and L of Table 1, FIG. 7A and FIG. 7B). Conversely, in all the experimental conditions examined, no detectable IFN-γ production was measured in T cells exposed to E40-digested gliadins (samples A-H of Table 1 and FIG. 7A and FIG. 7B).

Example 10—In Vitro Digestion of Gluten-Based Food Products (Bread)

In order to evaluate the efficiency of E40 enzyme as oral supplement to abolish the gluten intake in patients, in vitro oral-gastric digestion model was applied. Digestion step of bread (T. aestivum) was carried out under simulated physiological condition including oral mastication and presence of digestive gastric enzymes. Digestion time was selected according to the average time of chyme's transit in the gastric compartments. E40 enzyme preparation was added at beginning of the simulated gastric phase. The destruction of the T cell epitopes and toxic peptides was tested using monoclonal antibodies (Elisa-R5 competitive), according to the manufacturer's and to the AOAC guidelines.

Compared to E40-untreated samples, digested bread including E40 sample showed a drastic reduction of gluten content, well below to 20 ppm (Table 4).

TABLE 4

| | ppm |
|---|---|
| Bread digest sample | (ng/mL) |
| Oral-gastric digest | 303.0 |
| Oral-gastric-E40 digest | 16.2 |

Example 11—In Vitro Digestion of Peanut Proteins Incubated with Recombinant E40 (1:20, e:s)

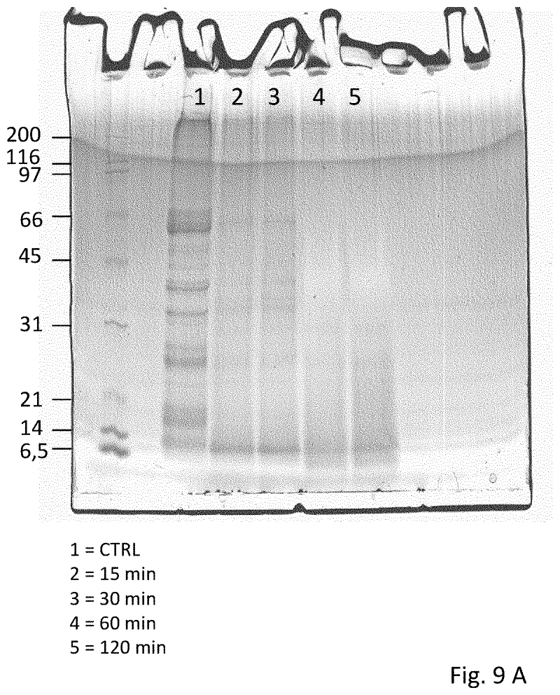

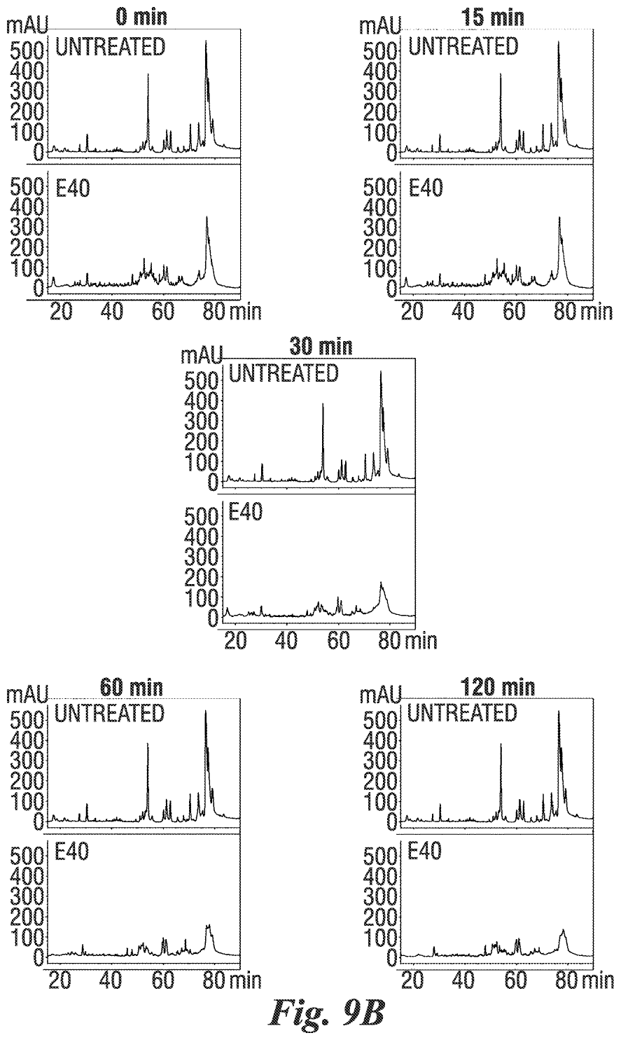

Degradation of peanuts proteins incubated in the presence of recombinant E40 has been assessed by SDS-PAGE and HPLC analysis. Results are shown in FIG. 9 (A: SDS-PAGE; B: HPLC analysis).

Example 12—Proteomic Analysis of Recombinant E40.

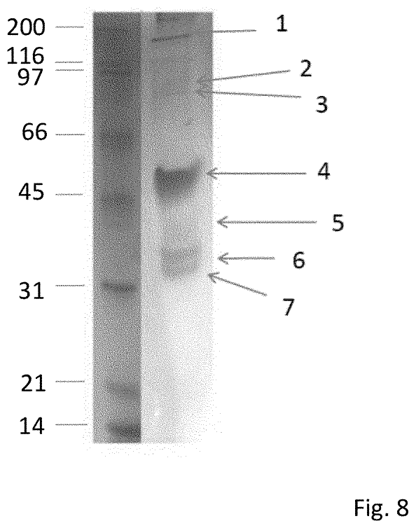

A detailed proteomic analysis of an enzyme preparation, obtained in S. lividans according to the method of the invention, was also performed. A sample of the enzyme preparation was run on SDS-PAGE (FIG. 8), then the lanes were excised and digested with trypsin, and the peptide mixture was analyzed through LS-MS/MS. Mass spectra were processed by means of the Proteome Discoverer software which revealed the identity of protein(s) within each gel band (Table 5).

TABLE 5

| | | | Nemysis | | | | | | | |
|---|---|---|---|---|---|---|---|---|---|---|
| | | | | | Best | Best | | | | |
| gel | | | Num | % | Disc | Expect | Protein | Protein | | |
| band | Rank | Acc # | Unique | Cov | Score | Val | MW | pI | Species | Protein Name |
| 1 | 1 | Q9ZBU3 | 11 | 21.0 | 8.35 | 7.4e−15 | 61099.2 | 6.5 | STRCO | Uncharacterized |
| | | | | | | | | | | protein |
| | 2 | Q9S2N0 | 9 | 38.3 | 6.04 | 1.4e−10 | 19219.8 | 4.7 | STRCO | Bacterioferritin |
| | 3 | Q54410 | 7 | 14.9 | 4.99 | 1.2e−8 | 58273.9 | 8.5 | STRLI | Tripeptidyl |
| | | | | | | | | | | aminopeptidase |
| 2 | 1 | A0A076MH05 | 18 | 34.4 | 7.34 | 5.5e−13 | 42792.4 | 6.3 | STRLI | Glycerophosphoryl |
| | | | | | | | | | | diester |
| | | | | | | | | | | phosphodiesterase |
| | 2 | Q54410 | 11 | 20.7 | 6.65 | 1.1e−11 | 58273.9 | 8.5 | STRLI | Tripeptidyl |
| | | | | | | | | | | aminopeptidase |
| 3 | 1 | O69935 | 22 | 29.4 | 5.87 | 2.9e−10 | 63258.4 | 6.5 | STRCO | Putative gamma- |
| | | | | | | | | | | glutamyltranspeptidase |
| | | | | | | | | | | (Putative |
| | | | | | | | | | | secreted protein) |
| | 2 | A0A076LZL8 | 14 | 27.2 | 6.76 | 6.5e−12 | 57422.7 | 6.5 | STRLI | Uncharacterized |
| | | | | | | | | | | protein |
| | 3 | Q54410 | 13 | 29.4 | 5.19 | 5.4e−9 | 58273.9 | 8.5 | STRLI | Tripeptidyl |
| | | | | | | | | | | aminopeptidase |
| | 4 | Q8CJI8 | 9 | 21.9 | 7.31 | 6.1e−13 | 42675.2 | 6.3 | STRCO | Putative |
| | | | | | | | | | | glycerophosphoryl |
| | | | | | | | | | | diester |
| | | | | | | | | | | phosphodiesterase |
| 4 | | Not identified | | | | | | | | |
| 5 | 1 | Q54344 | 4 | 9.2 | 6.57 | 1.5e−11 | 55425.9 | 5.5 | STRLI | Esterase |
| 6 | 1 | Q54344 | 13 | 24.0 | 7.73 | 1.0e−13 | 55425.9 | 5.5 | STRLI | Esterase |
| | 2 | D6EF89 | 10 | 35.9 | 6.15 | 8.6e−11 | 36594.8 | 6.6 | STRLI | Oxidoreductase |
| 7 | | Not identified | | | | | | | | |

Proteomic analysis confirmed that recombinant E40 protein migrates in the main band at −45 kDa (FIG. 8, band 4). SDS-PAGE also highlighted the presence of additional, though less intense bands. These bands were assigned to Streptomyces lividans proteins which have not been completely eliminated during the purification process. Some of these contaminants are proteases, but none of these has glutenase properties, confirming that recombinant E40 is the only protein active against gluten in the final enzyme preparation.

Example 13—Microbiological Assays on Enzyme Preparation Powders, in Fermentation and Down-Stream Process Steps

As known, S. lividans TK24 could potentially produce actinorhodin (ACT), undecylprodigiosin (UDP), and calcium-dependent antibiotic (CDA), especially when grown under fermentation conditions. Broth microdilution assay, standardized by the Clinical and Laboratory Standards Institute (CLSI) was then used to evaluate antibiotic presence in samples deriving from enzyme preparations obtained by the method of the invention. Antimicrobial activity has been tested against test microorganisms (test-mo), known to not show any antibiotic resistance: Staphylococcus aureus ATCC6538 (representative of Gram+); Escherichia coli L47 (Lepetit collection; representative of Gram-); Candida albicans L145 (Lepetit collection; representative of Yeast).

A two-fold serial dilution of the samples to be tested was performed in sterile 96-well microplates, then inoculated with the test-mo in their respective medium (100 μl/well final volume); each determination was carried out in triplicate. The inoculated microplates were then incubated for 18, 20 and 24 h for E. coli, S. aureus and C. albicans, respectively, at 35° C. Antimicrobial activity of samples of unknown “antimicrobial” concentration was measured as endpoint, that is the highest dilution that inhibits the test strain growth; it was determined by visual examination of the microplates with the aid of a magnifying mirror.

Assay control standard compounds were used: ciprofloxacin (activity against G+ and G−), daptomycin (only Gram+), amphotericin B (only C. albicans); furthermore, apramycin was tested (G+ and G−) since used in the fermentation process and also imidazole, as it may be used for eluting E40 from the Nickel resin.

Standard compounds were tested in the concentration range from 128 to 0.125 μg/ml, except for imidazole used in the concentration range from 0.35 mM to 250 mM; their antimicrobial activity expressed as MIC (minimal inhibitory concentration) is shown in the following Table 6.

TABLE 6

| | MIC (Minimal Inhibitory Concentration; μg/ml) | | |
|---|---|---|---|
| | S. aureus | E. coli | C. albicans |
| | ATCC6538 | L47 | L14 |
| Apramycin | 8 | 8 | >128 |
| Ciprofloxacin | 0.25 | 0.03 | >128 |
| Daptomycin | 0.5 | >128 | >128 |
| Amphotericin B | >128 | >128 | 0.5 |
| Imidazole (mM) | 125 | 125 | 3.9 |

Activity of three E40 enzyme preparation powder batches (F30, F34 and F35) was then verified: all the three batches analyzed did not show any antimicrobial activity at the highest concentration tested (sample dilution 1:4, corresponding to 2,5 mg/ml final powder concentration), showing that no antibiotics are present in the enzyme preparation. Control wells containing E40 powder samples in the not-inoculated media (that is without the test mo) did not show any microbial growth.

Antimicrobial activity of samples deriving from the a fermentation run of S.lividans TK24/pIJ86/e40 at 15L scale, with subsequent purification steps, according to the invention, has been further tested (Tables 7-9).

TABLE 7

| enzyme | ENDPOINT | | |
|---|---|---|---|
| preparation | S. aureus ATC6538 | | |
| purification step | replicates | | |
| HV supernatant | <1:2 | <1:2 | <1:2 |
| IMAC-E1 | <1:4 | <1:4 | <1:4 |
| DEAE-FT | <1:4 | <1:4 | <1:4 |
| TFF2 | <1:4 | <1:4 | <1:4 |
| SN 24 h | <1:4 | | |
| SN 50 h | <1:4 | | |
| SN 69 h | <1:4 | | |
| SN 90 h | <1:4 | | |
| SN 115 h | <1:4 | | |

| | |
|---|---|
| medium w/Apra | 1:4, about 12, 5 μg/ml Apra |

TABLE 8

| enzyme | ENDPOINT | | |
|---|---|---|---|
| preparation | E. coli L47 | | |
| purification step | replicates | | |
| HV supernatant | 1:2 | 1:2 | 1:2 |
| IMAC-E1 | 1:4 | 1:4 | 1:4 |
| DEAE-FT | <1:4 | <1:4 | <1:4 |
| TFF2 | <1:4 | <1:4 | <1:4 |
| SN 24 h | <1:4 | | |
| SN 50 h | <1:4 | | |
| SN 69 h | <1:4 | | |
| SN 90 h | <1:4 | | |
| SN 115 h | <1:4 | | |

| | |
|---|---|
| medium w/Apra | 1:4, about 12, 5 μg/ml apra |

TABLE 9

| enzyme | ENDPOINT | | |
|---|---|---|---|
| preparation | C. albicans L145 | | |
| purification step | replicates | | |
| HV supernatant | 1:2 | 1:2 | 1:2 |
| IMAC-E1 | 1:32 | 1:32 | 1:32 |
| DEAE-FT | 1:32 | 1:32 | 1:32 |
| TFF2 | <1:4 | <1:4 | <1:4 |
| SN 24 h | <1:4 | | |
| SN 50 h | <1:4 | | |
| SN 69 h | <1:4 | | |
| SN 90 h | <1:4 | | |
| SN 115 h | <1:4 | | |

| | |
|---|---|
| medium w/Apra | not tested |

The harvest (HV) culture supernatant (obtained by centrifugation at 17664g for 30 minutes), showed slight activity against E. coli and C. albicans that could be related to presence of undecylprodigiosin (UDP), as expected by its known activity pattern. The anticandida activity showed by the IMAC eluted E1 and the DEAE flow-through is surely due to imidazole; IMAC eluate low activity against E. coli is possibly still due to residual UDP presence. The final diafiltered TFF2 sample resulted to be free from any detectable antimicrobial activity. Regarding apramycin, its presence was not highlighted at the harvest of the culture although it is added to the productive medium at 50 μg/ml and its MIC results to be 8 μg/ml against both S.aureus and E.coli. This is probably due to decomposition and/or transformation by the action of enzymes.

Example 14—Testing Presence of Secondary Metabolites in E40 Enzyme Preparations and Down-Stream Intermediates

For safety assessment, the search for secondary metabolites produced by S. lividans TK24 is crucial. Dosing of the potential identified molecules in the fermentation supernatant as well in the formulated product is therefore required. For quantification purposes, HPLC/MS was selected as the best technique to evaluate the presence of ACT, UDP and Daptomycin (DAP, belonging to the CDA class). Extraction of antibiotics by solid phase (SPE) techniques was applied, with Diaion HP-(styrene-divinylbenzene) resin, allowing better quantitative analysis. Diaion HP-20 is generally used for the extraction of small molecules like secondary metabolites, pigments etc. with a wide range of polarity. Typically, resin is contacted with the aqueous solution in a ratio 1:20 v/v for 2 hours then transferred in a column, washed, and eluted with an increasing gradient of methanol in water. The eluted extracts are dried and then analyzed by HPLC/MS. A more appropriate column for small molecules and metabolites (Phenomenex Luna C18, 5 μm, 2.1×250 mm) was used. E1 (50% methanol), E2 (100% methanol) and E3 fractions (methanol/isopropanol 90:10) are collected. Daptomycin is eluted in E1 and E2, while Undecylprodigiosine (UDP) is eluted in E2 and E3. Recovery of Daptomycin is quantitative, while UDP has the tendency to bind tightly to resins, filters and membranes so the recovery was partial (20-25%).

Since Apramycin does not bind to HP-20, an alternative method of extraction was applied for Apramycin. Amberlite IRC 50 weak cationic resin in NH4 form was tested and showed to bind Apramycin quantitatively in a single column transit step. Then, elution with 0.1M ammonium hydroxide gave a complete recovery.

The Luna C18 HPLC column was used as default for extracted samples with a gradient of phase B from 5 to 90% in 30 min and flow rate 1,0 ml/min.

Final tests with 50 mg of E40 enzyme preparations in powder (batches F-30, F-34, F-35) were completed and gave the following results (Table 10):

TABLE 10

| E40 | | | MIN. PPM | | | |
|---|---|---|---|---|---|---|
| POWDERS | ANALYZED | LIMIT OF | (mg/kg) | | | |
| by SPE | FRACTION | DETECTION (ng) | * | F-30 | F-34 | F-35 |
| APRAMYCIN | FT | 17.5 | 0.875 | <min. | <min. | <min. |
| | | | | ppm | ppm | ppm |
| UDP | E2 + E3 | 0.1 | 0.005 | <min. | <min. | <min. |
| | | | | ppm | ppm | ppm |
| DAPTOMYCIN | E1 + E2 | 0.6 | 0.03 | <min. | <min. | <min. |
| | | | | ppm | ppm | ppm |

(* based on analysis of 20 mg final powder)

These data confirm that none of the analyzed antibiotics can be found in measurable amounts in any of the three E40 enzyme preparation batch powders.

With the application of this methodology, the detection of the same antibiotics in the fermentation culture and in the subsequent downstream purification steps was monitored. The fermentation culture named F-46 was setup with this purpose. The fermentation culture underwent the following purification steps, according to a preferred embodiment of the method of the invention:

1. Centrifugation (30′ at 17664 g) to recover the sample HV-Supernatant

2. Clarification (Opticap PES capsules 1.0 and 0.5 micron nominal size)

3. TFF1 concentration (with 10kD cutoff regenerated cellulose cassette)

4. IMAC Ni-based affinity chromatography

5. Depigmentation by ion exchange with DEAE resin

6. TFF2 concentration and diafiltration (10 kDa cutoff)

7. Lyophilization after addition of excipients to obtain the final powder.

Samples from each step were analyzed by HPLC-MS, searching for the above antibiotics by molecular weight. Small volumes were injected (12.5 microliters). Only UDP was found in measurable amounts. No trace of the others was detected.

Regarding Undecylprodigiosine (UDP), the concentration in each step was quantified by HPLC-MS (Table 11). As expected, UDP is successfully removed during the downstream process:

TABLE 11

| | | | | | STEP BY |
|---|---|---|---|---|---|
| | | | | | STEP |
| UDP in F-46 | | CONC | TOTAL | % | REMOVAL |
| SAMPLES | VOL ML | ng/ml | μg | RESIDUE | % |
| 1-HV- | 11260 | 116.47 | 1311.50 | 100.00 | — |
| Supernatant | | | | | |
| 2-Clarifled | 10500 | 56.34 | 591.52 | 45.10 | 54.90 |
| w Opticap | | | | | |
| 3a-TFF1 | 1080 | 461.46 | 498.37 | 38.00 | 15.75 |
| retentate | | | | | |
| 3b-TFF1 | 8040 | 2.04 | 16.42 | 1.25 | — |
| permeate | | | | | |
| 4a-IMAC | 270 | 17.73 | 4.79 | 0.37 | 99.04 |
| eluted E1 | | | | | |
| pH 6.3 | | | | | |
| 4b-IMAC | 1080 | 818.10 | 883.55 | 67.37 | — |
| flowthrough | | | | | |
| 5-DEAE | 254 | 6.72 | 1.71 | 0.13 | 64.34 |
| depigmentation | | | | | |
| 6-TFF2 | 61 | 1.31 | 0.08 | 0.01 | 95.32 |
| diafiltration | | | | | |

In conclusion, the enzyme preparation powders comprising E40, obtained by a method according to the invention, do not contain measurable amounts of antibiotics.

Example 15

Two new 15 L fermentations according to the invention were performed, starting from a seed culture in flasks incubated for about 42 hours. The two cultures, named F47 and F48, have been harvested after 99 and 94 hours under fermentation conditions respectively. The harvested cultures were submitted to centrifugation (Beckmann J-2-21; Rotor JA-10; 10000 rpm/17700g, 15 min) and filtration on paper, and the supernatant was then microfiltered (clarification step) in three subsequent stages (starting from a 1 μm filtration, up to a 0.3 μm filtration). Ultrafiltration (TFF1) of the clarified solution was then applied, followed by IMAC-Nickel affinity chromatography of the ultrafiltration permeates, performed with Ni Sepharose 6 FastFlow (GE Healthcare) resin as described before. An optional step of DEAE anion exchange depigmentation chromatography then followed:elution fractions from IMAC were adjusted to pH 6.3 with formic acid, transferred into a DEAE Sepharose CL-6B column equilibrated with 20 mM ammonium formate pH 6.3 and collected by gravimetry. Ultrafiltration system for smaller volumes (TFF2-diafiltration) was setup (nominal MW limit: 10 kD) for ultrafiltration of elution fraction from DEAE, then desalting solution prepared with ammonium formate 10 mM pH 6.3 was added in portions in order to remove the remaining imidazole. The solution may become cloudy, requiring centrifugation to discard a precipitate. The removed solid does not include recombinant E40. This can be avoided by keeping the solution more diluted in this step. The final TFF2 solution obtained was added with 3:1 mannitol and trehalose solution, corresponding to 8 micrograms of total sugar per AU, then it was freeze-dried.

Final powder yield was 1437 mg. A sample of the final powder was dissolved at 10 mg/ml in a 20% EtOH aqueous solution and checked for enzymatic activity and protein content, and then submitted to SDS-page.

BCA total protein content assay is around 14% in the batch final powder (100% if excluding added sugars).

The production of E40 is of about 25 mg/L of supernatant (about 20 mg/L fermentation volume).

Microbiological activity of the above preparations was tested at the higher concentration of 5 mg/ml (stock solution at 10 mg/ml in dH20) and after two-fold serial dilutions. Activity of the F47/F48 - generated powder is shown in Table 12: no activity was shown at the highest concentration tested against any of the test-mo analyzed.

TABLE 12

| | MIC (Minimal Inhibitory Concentration; mg/ml) | | | | | | | | |
|---|---|---|---|---|---|---|---|---|---|
| sample | S. aureus ATCC6538 | | | E. coli L47 | | | C. albicans L145 | | |
| replicate | #1 | #2 | #3 | #1 | #2 | #3 | #1 | #2 | #3 |
| pwd F47/48 | >5 | >5 | >5 | >5 | >5 | >5 | >5 | >5 | >5 |

This result confirms that the final E40 preparation is free from detectable antibiotics.

Example 16

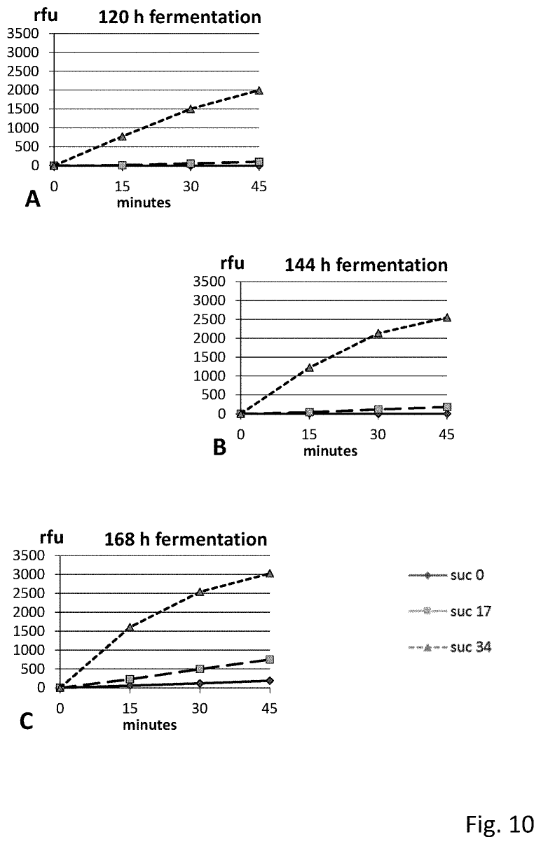

The recombinant S. lividans TK24/p1J86/e40 host cells of Example 2 were inoculated in Erlenmeyer flasks (500 ml) containing 100 ml of Medium V (as described above) added with Apramycin 50 mg/L, and incubated on a rotatory shaker at 200 rpm at 30° C. After 4 days of growth, the culture was used to inoculate three Erlenmeyer flasks (500 ml) containing 100 ml of Medium P, as described above, containing Sucrose at 340 g/L (suc 34), or at 170 g/L (suc 17) or not containing sucrose (suc 0), respectively. All the flasks were added with Apramycin 50 mg/L and incubated on a rotatory shaker at 200 rpm at 30° C. for up to 7 days. E40 production was monitored once a day after 120h of fermentation by measuring the enzymatic activity of the culture supernatants, obtained by centrifugation at 16,000 g for 6 min. Proteolytic activity of supernatant samples, diluted 1:81 in an assay buffer just before testing, was assessed in microtiter wells toward the fluorogenic substrate suc-Ala-Ala-Pro-Phe-AMC (Bachem L-1465; 200 μM) in citrate 0.1M—phosphate 0.2M reaction buffer, pH5 (20 μL sample in 200 μL total reaction volume). Incubation was performed at 37° C. for 30 minutes. The released AMC was measured with a Fusion micro-plate reader (Perkin Elmer Italia SpA, Monza, Italia) at excitation wavelength of 360 nm and emission wavelength of 460 nm. Proteolytic activity was determined as linear increase of relative fluorescence units produced over time (rfu/minute) in the interval time 0-15 minutes. Results are shown in FIG. 10.

Significant impact on E40 production is given by the presence of sucrose in the medium used for fermentation, as shown in FIG. 10, panels A-C. Proteolytic activity is evident for the cultures grown in medium P with 34% sucrose, while it is substantially absent in the medium without sucrose. In the presence of 17% sucrose, low activity is shown, even after 168 hours of fermentation. Results are reported for each sample in table 13 (relative fluorescence units is expressed per minute by 1 mL supernatant (rfu/min/mL) in the incubation interval 0-15 minutes).

TABLE 13

| 0-15′ | | | |
|---|---|---|---|
| interval | fermentation time (hours) | | |
| rfu/min/mL | 120 | 144 | 168 |
| suc 0 | 0 | 0 | 183 |
| suc 17 | 47 | 145 | 763 |
| suc 34 | 2583 | 4085 | 5353 |

Example 17

Saccharomyces cerevisiae (S. cerevisiae) type strain X4004-3A was used as host for heterologous expression of E40. Two different synthetic E40 genes were designed that differ for the presence or absence of the sequence coding for the pro-enzyme; in both genes the native signal secretion sequence of E40 was replaced with the modified α-factor prepro-leader sequence²⁰. Furthermore, in both the synthetic genes the codon usage was optimized according to that of S. cerevisiae by using the Codon Optimization Tool software. Sequences corresponding to Xbal and HindIII restriction sites Both genes were also added. The resulting genes, one coding for the inactive precursor (proE40, SEQ ID NO: 13) and the other for the mature enzyme (matE40, SEQ ID NO:14) were cloned into either the inducible pEMBL and constitutive pVT-U expression plasmids, both carrying the URA3 gene for auxotrophic selection in S. cerevisiae^{19,21} and then introduced into ElectroMAX DH10B E. coli cells by electroporation method. Transformants were selected on ampicillin containing LB agar plates (100 μg/mL) incubated overnight at 37° C. The correctness of constructs was confirmed by sequencing. Transformants were selected on ampicillin-containing LB agar plates (100 μg/mL) incubated overnight at 37° C. The pEMBL/E40 and pVT-U/E40 plasmids were transferred to the host S. cerevisiae strain X4004-3A by employing the lithium acetate method (Elble, 1992). Cells were plated on selective plates (medium lacking uridine) and incubated at 30° C. for 5-6 days.

A single colony from the S. cerevisiae clones containing E40 genes was picked from the selective plate, inoculated in 100 ml of minimal medium (0.68% yeast nitrogen base, YNB, 2% glucose, 50 mg/L of amino acid L-Lys-L-Met-L-Trp, 67 mM potassium phosphate, pH 6.0) for 3 days at 30° C.

An aliquot of cells was removed and inoculated into a final volume of 50 mL of minimal medium in a 500-mL flask starting from OD600nm=0.25. Incubation proceeded until OD600nm reached the value 0.5 (6 hours), then 8-mL were inoculated into 500 mL flask containing 72 mL of expression medium (10 g/L yeast extract, 20 g/L peptone and 2% glucose for pVT-U/E40 plasmid or 2% galactose for the pEMBL/E40 inducible expression plasmid) for 10 days. For protein expression trials, cells were grown at 30° C. and harvested at different times.

The expression of recombinant E40 was checked in culture supernatants by an activity assay using the synthetic substrate suc-Ala-Ala-Pro-Phe-pNA, following the absorbance at 410 nm during the course of incubation at pH 5 and 37° C. The enzymatic activity was expressed as absorbance units (au) per minute per ml. The maximal activity was detected in the supernatant of cells transformed with the plasmid pVT-U/matE40 and grown for days: a value of 0.9 au/min/mL was shown. A parallel fermentation trial of S. cerevisiae transformed with the four constructs was performed, resulting in similar expression levels (0.73 ΔAbs min⁻¹ mL⁻¹). On the basis of these results a Western blot analysis was performed on the supernatants of cells transformed with plasmids pVT-U/matE40 and pEMBL/matE40 collected at different times. Western blot analysis showed that when the pEMBL-matE40 plasmid was used, the protein has been expressed after 6 days of growth; for the pVT-U/matE40 plasmid, the protein expression was detected after 3 days. S. cerevisiae can thus be used as recombinant producer of E40. However, the modest expression of recombinant E40 obtained (less than 1 mg E40 for ml of supernatant) renders this production system currently not suitable for industrial exploitation.

In conclusion, the recombinant expression of E40 in S. lividans host cells according to the method of the invention results in the secretion of active E40. All the chemical-physical characteristics and biological properties of the native Actinoallomurus A8 form are maintained by the recombinant glutenase, e.g. stability and activity at post-prandial gastric pHs, resistance to pepsin digestion, high efficiency in the extensive degradation of the most immunogenic peptide of a-gliadin, such as the Pro-Gln enriched 33-mer peptide (SEQ ID NO: 12), as well as of whole gliadin proteins.

The enzyme preparation obtained by the method of the invention thus provides high amounts of active recombinant E40 and it is furthermore substantially free of antibiotics, being thus suitable for human use, preferably after proper formulation as pharmaceutical formulation or food supplement.

Example 18

A synthetic gene coding for E40 in its native mature form (matE40, SEQ ID NO:14) was cloned into an expression vector for successful secretion in the yeast Pichia pastoris, bearing AOX1 promoter variants for methanol-induced expression (mut^{s}) and the pre-pro sequence, without the Glu-Ala (EAEA) repeats, of the alpha-mating factor from Saccharomyces cerevisiae, for directing the secretion in the culture medium. Zeocin resistance gene was also present in the vector as selection marker. Correct insertion of the target gene into the expression vector was checked by restriction pattern analysis, and authenticity of the gene sequences was confirmed by sequencing (LGC Genomics, Berlin, Germany). Purified plasmid at a concentration of about 1 μg/μL was used for transformation into muts and muts-PDI (overproducing Protein Disulphide-Isomerase) P. pastoris strains, the latter to facilitate correct folding of the protein. Several individual colonies per strain (2 muts and 2 muts-PDI strains bearing the E40-expressing vector and the respective muts and muts-PDI mock strains) were picked and inoculated into single wells of 96-deep well plates filled with complex cultivation medium. Production evaluation was performed under methanol-inducible conditions using four different conditions:

Complex medium at pH6.0

Complex medium at pH6.0 supplemented with sucrose (34% final)

Complex medium at pH7.0

Complex medium at pH7.0 supplemented with sucrose (34% final)

After an initial growth phase to generate biomass, expression was induced by addition of an optimized liquid mixture containing a defined concentration of methanol. At this time, a control sample consisting of purified E40 as reference material was spiked into several wells containing the mock strains at a final concentration of 200 μg/mL. At defined points of time, further induction with methanol was performed.

Sampling was done after 50 hours as well as after 96 hours from methanol induction time (end of process). Supernatant samples, obtained after centrifugation, were immediately adjusted to contain 20% EtOH; samples taken at 50 hours were frozen to be submitted to activity assessment together with those sampled at 96 hours, which were directly tested.

Supernatant samples were subjected to activity measurements in undiluted form, whereas control samples of mock strains supernatants with spiked E40 reference material were diluted 1:20 with assay buffer before performing the activity assay.

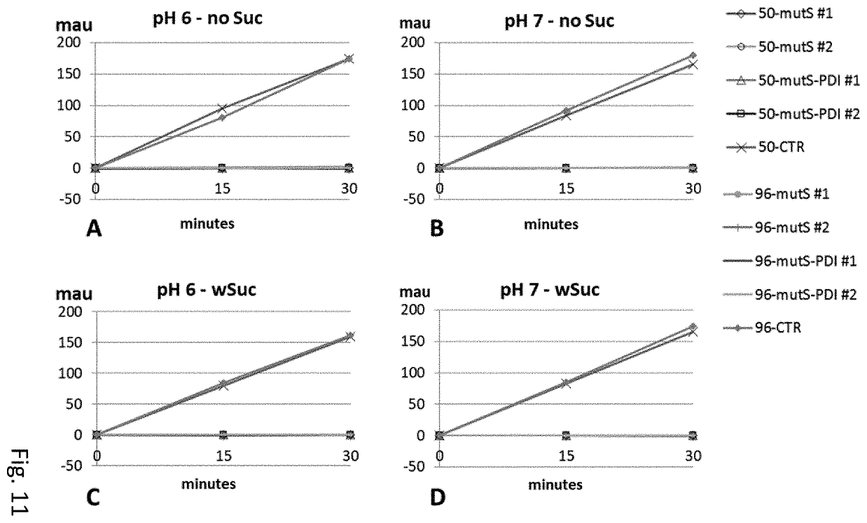

Activity was assessed in microtiter plates using the chromogenic substrate suc-Ala-Ala-Pro-Phe-pNA (Bachem L-1400; 200 μM) in citrate 0.1M-phosphate 0.2M reaction buffer, pH5 (20 μL sample in 200 μL total reaction volume). Incubation was performed at 37° C. for 30 minutes. Proteolytic activity was determined as linear increase of absorbance (read at 410 nm wavelength) during time, and expressed as milli-absorbance units per minute (mau/minute). Results are shown in FIG. 11.

Control samples (50 CTR and 96 CTR in FIG. 11) show significant proteolytic activity in all conditions and backgrounds (5.3 to 6.0 milli-absorbance units per minute for the samples tested, having E40 concentration of 8.3 mg/L) whereas no increase in absorbance is given by supernatant samples from any of the other cultures (<0.07 mau/min), corresponding to E40 concentrations ≤0.1 mg/L. Results are reported in table 14.

TABLE 14

| | 50 h post induction | | | | 96 h post induction | | | |
|---|---|---|---|---|---|---|---|---|
| milli- | pH 6 | | pH 7 | | pH 6 | | pH 7 | |
| au/ min | no Suc | w Suc | no Suc | w Suc | no Suc | w Suc | no Suc | w Suc |
| mutS #1 | 0.009 | 0 | 0.001 | 0 | 0.065 | 0.021 | 0.032 | 0 |
| mutS #2 | 0.016 | 0 | 0.015 | 0 | 0.021 | 0 | 0.02 | 0 |
| mutS- PDI #1 | 0 | 0 | 0 | 0 | 0 | 0 | 0 | 0 |
| mutS- PDI #2 | 0 | 0 | 0 | 0 | 0 | 0 | 0 | 0 |
| CTR | 5.8 | 5.3 | 5.5 | 5.5 | 5.8 | 5.4 | 6 | 5.8 |

Example 19

Escherichia coli BL21 Star (DE3) host cells were transformed with recombinant pET28b expression plasmid bearing E40 sequence as reported in WO2013083338. Production of E40 by transformed cells was induced by addition of 0.2 μM isopropyl β-D-thiogalactoside (Sigma) and expression of the desired protein was obtained (see FIG. 4 of WO2013083338).

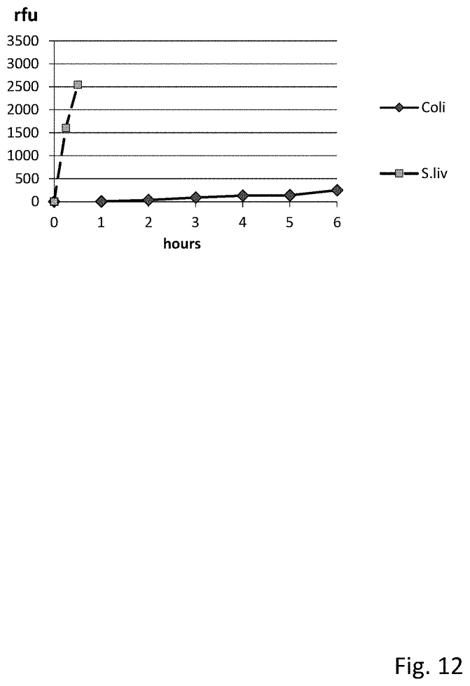

50 mL of E. coli cell culture expressing the recombinant E40 were submitted to lysis according to the Invitrogen protocol for native lysis, based on lysozyme activity, as described in WO2013083338; the whole pellet from 50 mL culture was resuspended in 8 mL of lysis buffer solution. The crude extract obtained after lysis was tested for proteolytic activity (Ammonium Acetate buffer 50 mM, pH; 37° C.) onto the substrate Succinyl-Ala-Ala-Pro-Phe-AMC. The activity is expressed as rfu produced per minute by 1 mL of the E40-producing E. coli culture is reported in FIG. 12. The activity of E40 produced in E. coli is very low, compared to that obtained for E40 produced in S. lividans under conditions according to the invention (54 rfu/min/mL with E.coli compared to >5300 rfu/min/mL with S. lividans; see FIG. 12).

### REFERENCES

1. Abadie, V., Sollid, L. M., Barreiro, L. B., Jabri, B. Integration of genetic and immunological insights into a model of celiac disease pathogenesis. Annu Rev Immunol. 29, 493-525 (2011).

2. Wieser, H. Chemistry of gluten proteins. Food Microbiol. 24,115-9 (2007).

3. Hausch, F., Shan, L., Santiago, N. A., Gray, G. M., Khosla, C. Intestinal digestive resistance of immunodominant gliadin peptides. Am. J. Physiol. Gastrointest. Liver Physiol. 283, G996-1003 (2002).

4. Gianfrani, C., Camarca, A., Mazzarella, G., Di Stasio, L., Giardullo, N., Ferranti, P., Picariello, G., Rotondi Aufiero, V., Picascia, S., Troncone, R., Pogna, N., Auricchio, S., Mamone, G. Extensive in vitro gastrointestinal digestion markedly reduces the immune-toxicity of Triticum monococcum wheat: implication for celiac disease. Mol. Nutr. Food Res. 59,1844-54 (2015).

5. Shan, L., Molberg, 0., Parrot, I., Hausch, F., Filiz, F., Gray, G. M., Sollid, L. M., Khosla, C. Structural basis for gluten intolerance in celiac sprue. Science 297, 2275-2279 (2002).

6. Kupper, C. Dietary guidelines and implementation for celiac disease. Gastroenterology 128, S121-7 (2005).

7. Ribeiro, M., Nunes, F. M., Rodriguez-Quijano, M., Carrillo, J. M., Branlard, G., Igrejas, G. Next-generation therapies for celiac disease: The gluten-targeted approaches. Trends in Food Science & Technology, 5, 56-71 (2018).

8. Dressman, J. B., Berardi, R. R., Dermentzoglou, L. C., Russell, T. L., Schmaltz, S. P., Barnett, J. L., Jarvenpaa, K. M. Upper gastrointestinal (GI) pH in young, healthy men and women. Pharm. Res. 7, 756-61 (1990).

9. Gardner, J. D., Ciociola, A. A., Robinson M. Measurement of meal-stimulated gastric acid secretion by in vivo gastric autotitration. J. Appl. Physiol. 92, 427-34 (2002).

10. Moron, B., Cebolla, A., Manyani, H., Alvarez-Maqueda, M., Megias, M., Thomas Mdel, C., Lopez, M. C., Sousa, C. Sensitive detection of cereal fractions that are toxic to celiac disease patients by using monoclonal antibodies to a main immunogenic wheat peptide. Am. J. Clin. Nutr. 87, 405-14 (2008).

11. Sollid, L. M., Qiao, S. W., Anderson, R. P., Gianfrani, C., Koning, F. Nomenclature and listing of celiac disease relevant gluten T-cell epitopes restricted by HLA-DQ molecules. Immunogenetics 64, 455-60 (2012).

12. Anne, J., Vrancken, K., Van Mellaert, L., Van Impe, J., & Bernaerts, K. Protein secretion biotechnology in Gram-positive bacteria with special emphasis on Streptomyces lividans. Biochimica et Biophysica Acta (BBA)-Molecular Cell Research. 1843(8), 1750-1761 (2014).

13. Kieliszek, M., & Misiewicz, A. Microbial transglutaminase and its application in the food industry. A review. Folia Microbiologica. 59, 241-250 (2013).

14. Flett, F., Mersinias, V., Smith, C. P. High efficiency intergeneric conjugal transfer of plasmid DNA from Escherichia coli to methyl DNA-restricting streptomycetes. FEMS Microbiol. Lett. 155, 223-229 (1997).

15. Kieser, T., Bibb, M. J., Buttner, M. J., Chater, K. F., Hopwood, D. A. Practical Streptomyces in Genetics, (John Innes Foundation, 2000).

16. Jena, S., Suryanarayanan, R., Aksan, A. l Mutual Influence of Mannitol and Trehalose on Crystallization Behavior in Frozen Solutions. Pharm Res. 33, 1413-25 (2016).

17. Kriegler M. “Gene Transfer and Expression: A Laboratory Manual” Stockton Press, N.Y., 1990.

18. Christian Rückert, Andreas Albersmeier, Tobias Busche, Sebastian Jaenicke, Anika Winkler, Ólafur H. Friōjónsson, Guōmundur Óli Hreggviōsson, Christophe Lambert, Daniel Badcock, Kristel Bernaerts, Jozef Anne, Anastassios Economou, Jörn Kalinowski “Complete genome sequence of Streptomyces lividans TK24” Journal of Biotechnology, 2015 (Volume 199, Pages 21-22)

19. Cesarini G, Murray J A H. Plasmid vectors carrying the replication origin of filamentous single-stranded phages. In: Setlow J K, editor. Genetic engineering, principles and methods. New York: Plenum Press, 1987, pp. 135-154.

20. Tonin F, Melis R, Cordes A, Sanchez-Amat A, Pollegioni L, Rosini E. Comparison of different microbial laccases as tools for industrial uses. N Biotechnol, 2016, 33: 387-398.

21. Vernet T, Dignard D, Thomas D Y. A family of yeast expression vectors containing the phage fl intergenic region. Gene, 1987, 52: 225-233.

1-11. (canceled)

12. A method for producing an enzyme preparation comprising at least one recombinant Actinoallomurus endopeptidase having glutenase activity, the method comprising in series:
a) culturing a recombinant Streptomyces lividans host cell in a culture medium under fermentation conditions, said recombinant host cell comprising a recombinant expression vector for heterologous expression of the at least one recombinant Actinoallomurus endopeptidase, the recombinant expression vector comprising a polynucleotide encoding for said at least one recombinant Actinoallomurus endopeptidase operably linked to regulatory sequences capable of directing the expression of said at least one recombinant Actinoallomurus endopeptidase in the recombinant host cell;
wherein the culture medium comprises at least 30% (wt/vol) sucrose;
b) recovering the supernatant of the culture medium comprising the at least one recombinant Actinoallomurus endopeptidase; and
c) purifying from said supernatant the enzyme preparation comprising the at least one recombinant Actinoallomurus endopeptidase.

13. The method of claim 12, wherein the recombinant Actinoallomurus endopeptidase having glutenase activity is selected from the group consisting of:
endopeptidase 40 (E40) of sequence comprising SEQ ID NO: 1; a biologically active fragment of E40; a naturally occurring allelic variant of E40; and an endopeptidase of sequence having at least 60%, 70%, 80%, 90% or 95% of identity to SEQ ID NO: 1.

14. The method of claim 12, wherein the fermentation conditions comprise culturing the recombinant host cell at a temperature between 28° C. and 30° C.

15. The method of claim 12, wherein in step b) culturing under fermentation conditions is carried on for at least 48 hours.

16. The method of claim 15, wherein in step b) culturing under fermentation conditions is carried on for at least 72 hours.

17. The method of claim 12, wherein the recombinant Streptomyces lividans host cell is of the TK24 strain.

18. The method of claim 12, wherein the enzyme preparation purified in step c) comprises the mature form of E40 of sequence comprising or consisting of SEQ ID NO: 1 or a histidine tagged E40 of sequence comprising or consisting of SEQ ID NO: 2.

19. The method of claim 12, wherein the enzyme preparation purified in step c) is in powder form.

20. The method of claim 12, wherein the recombinant expression vector comprises a polynucleotide encoding for E40 of sequence comprising SEQ ID NO: 1; wherein the polynucleotide is of sequence SEQ ID NO:5 or SEQ ID NO: 6, or of sequence having at least 60%, 70%, 80%, 90% or 95% of identity to SEQ ID NO: 5 or SEQ ID NO: 6.

21. The method of claim 12 wherein the recombinant expression vector is recombinant pIJ86.

22. A recombinant pIJ86 expression vector, comprising a polypeptide having sequence comprising SEQ ID NO: 5 or SEQ ID NO:6.

23. A recombinant Streptomyces lividans host cell comprising the recombinant expression vector of claim 22.

24. The recombinant Streptomyces lividans host cell of claim 23, being of strain DSM 33207.

25. A formulation comprising as the active proteolytic ingredient the enzyme preparation obtained by the method of claim 12 or an isolated recombinant Actinoallomurus endopeptidase having glutenase activity, isolated from the supernatant of the culture medium recovered in step c) of claim 12, the formulation being a food or a food supplement or a pharmaceutical formulation, or a flour that has been put in contact with the enzyme preparation or with the isolated recombinant Actinoallomurus endopeptidase.

26. The formulation of claim 25, wherein the isolated recombinant Actinoallomurus endopeptidase is selected from the group consisting of: endopeptidase 40 (E40) of sequence comprising SEQ ID NO: 1; a biologically active fragment of E40; a naturally occurring allelic variant of E40; and an endopeptidase of sequence having at least 60%, 70%, 80%, 90% or 95% of identity to SEQ ID NO: 1.

27. A method of treating and/or preventing a disorder selected from the group consisting of: celiac disease, a disorder associated to celiac disease, non-celiac gluten sensitivity, and allergy or intolerance to nuts and/or peanuts, by administering to a subject in need thereof an effective amount of the formulation of claim 25.

28. The method of claim 27 wherein the disorder is associated with intolerance to gliadin peptide of sequence SEQ ID NO:6.


SEQUENCE LISTING (14 sequences)

SEQ ID NO: 1 - protein, 325 aa, Actinoallomurus
AAPSGLSPANLQSAYKLPSSTAGSGQTVAIVDAYDAPTAEADLNVYRSQFGLGACTTANGCFKKVDQNGGTSYPRKDGGWAQEISLDLDMVSAVCPNCKIVLVEAKTNSFANLGTAENTAASLANVISNSYGGSDASDASYGSYYNHPGKAITVSSGDAGYGVEYPASSHYVTAVGGTSLRTASTSRGWSETAWSGAGSGCSAYNTALSGQSGLTGCSRRAVADVSAVADPATGVAVYDSTAYQGQSGWMVFGGTSVAAPIIGGVYGLAANAASIDNNYPYAHTSSLFDVTSGSNGTCTTTKWCTAGTGWDGPTGLGTPNGTGAF

SEQ ID NO: 2 - protein, 332 aa, Actinoallomurus
AAPSGLSPANLQSAYKLPSSTAGSGQTVAIVDAYDAPTAEADLNVYRSQFGLGACTTANGCFKKVDQNGGTSYPRKDGGWAQEISLDLDMVSAVCPNCKIVLVEAKTNSFANLGTAENTAASLANVISNSYGGSDASDASYGSYYNHPGKAITVSSGDAGYGVEYPASSHYVTAVGGTSLRTASTSRGWSETAWSGAGSGCSAYNTALSGQSGLTGCSRRAVADVSAVADPATGVAVYDSTAYQGQSGWMVFGGTSVAAPIIGGVYGLAANAASIDNNYPYAHTSSLFDVTSGSNGTCTTTKWCTAGTGWDGPTGLGTPNGTGAFGHHHHHH

SEQ ID NO: 3 - protein, 398 aa, Actinoallomurus
MSRRVTGTILGGLILAMVPFLSTAANAAPQAAPASVSHPFHHSCATVKPGRASCNALVRSDIAQSAATLAHQAAAPSGLSPANLQSAYKLPSSTAGSGQTVAIVDAYDAPTAEADLNVYRSQFGLGACTTANGCFKKVDQNGGTSYPRKDGGWAQEISLDLDMVSAVCPNCKIVLVEAKTNSFANLGTAENTAASLANVISNSYGGSDASDASYGSYYNHPGKAITVSSGDAGYGVEYPASSHYVTAVGGTSLRTASTSRGWSETAWSGAGSGCSAYNTALSGQSGLTGCSRRAVADVSAVADPATGVAVYDSTAYQGQSGWMVFGGTSVAAPIIGGVYGLAANAASIDNNYPYAHTSSLFDVTSGSNGTCTTTKWCTAGTGWDGPTGLGTPNGTGAF

SEQ ID NO: 4 - protein, 405 aa, Actinoallomurus
MSRRVTGTILGGLILAMVPFLSTAANAAPQAAPASVSHPFHHSCATVKPGRASCNALVRSDIAQSAATLAHQAAAPSGLSPANLQSAYKLPSSTAGSGQTVAIVDAYDAPTAEADLNVYRSQFGLGACTTANGCFKKVDQNGGTSYPRKDGGWAQEISLDLDMVSAVCPNCKIVLVEAKTNSFANLGTAENTAASLANVISNSYGGSDASDASYGSYYNHPGKAITVSSGDAGYGVEYPASSHYVTAVGGTSLRTASTSRGWSETAWSGAGSGCSAYNTALSGQSGLTGCSRRAVADVSAVADPATGVAVYDSTAYQGQSGWMVFGGTSVAAPIIGGVYGLAANAASIDNNYPYAHTSSLFDVTSGSNGTCTTTKWCTAGTGWDGPTGLGTPNGTGAFGHHHHHH

SEQ ID NO: 5 - DNA, 1197 bp, Actinoallomurus
atgtcacgacgcgtgaccgggaccatactgggcgggttgatcctcgccatggtccccttcctttccaccgcggccaacgccgcaccccaggccgcgccggcttccgtctcccacccgttccaccactcctgcgccacggtgaagccgggtcgggcgagctgcaatgccctcgtacgcagcgacatcgcccagagcgcggcgaccctcgcgcaccaagcggccgccccatccgggctctcgccggccaacctgcagagcgcctacaagctgccgtcctccacggccggatccggccagaccgtcgcgatcgtcgacgcctatgacgccccgaccgccgaagcggacttgaacgtgtaccgaagccagttcggactcggcgcgtgcacgaccgccaacggctgtttcaagaaggtcgaccagaacggcggcacgtcctatccgaggaaggacggcggctgggcgcaggagatctccctggacctcgacatggtctccgcggtctgccccaactgcaagatcgttctcgtcgaggcgaagaccaactcgttcgccaacctgggtaccgccgagaacaccgcggcgagtctcgcgaacgtcatcagcaacagctacggcggctcggacgcctctgacgcgagctatggctcgtactacaaccacccgggcaaggccatcacggtcagctccggcgacgccggctacggcgtggagtacccggcctcgtcccactacgtgaccgccgtcggcggcacctcgctgcgcaccgcgagcaccagccgcggctggagcgagaccgcgtggagcggcgcgggcagtggctgctcggcctacaacaccgcgctgtccggccagtccggcctcaccggctgctcccggcgcgccgtcgccgacgtctccgccgtggccgacccggccaccggcgtcgccgtctacgacagcacggcctaccagggccagagcggctggatggtcttcggcggcaccagcgtcgccgcaccgatcatcggtggcgtgtacggcctcgccgccaacgccgcgagcatcgacaacaactacccctacgcccacaccagctcgctcttcgacgtcacgtcgggcagcaacggcacctgcaccaccaccaagtggtgcaccgccggcaccggctgggacggccccaccggcctcggaacgccgaacggcaccggagccttctga

SEQ ID NO: 6 - DNA, 1218 bp, Actinoallomurus
atgtcacgacgcgtgaccgggaccatactgggcgggttgatcctcgccatggtccccttcctttccaccgcggccaacgccgcaccccaggccgcgccggcttccgtctcccacccgttccaccactcctgcgccacggtgaagccgggtcgggcgagctgcaatgccctcgtacgcagcgacatcgcccagagcgcggcgaccctcgcgcaccaagcggccgccccatccgggctctcgccggccaacctgcagagcgcctacaagctgccgtcctccacggccggatccggccagaccgtcgcgatcgtcgacgcctatgacgccccgaccgccgaagcggacttgaacgtgtaccgaagccagttcggactcggcgcgtgcacgaccgccaacggctgtttcaagaaggtcgaccagaacggcggcacgtcctatccgaggaaggacggcggctgggcgcaggagatctccctggacctcgacatggtctccgcggtctgccccaactgcaagatcgttctcgtcgaggcgaagaccaactcgttcgccaacctgggtaccgccgagaacaccgcggcgagtctcgcgaacgtcatcagcaacagctacggcggctcggacgcctctgacgcgagctatggctcgtactacaaccacccgggcaaggccatcacggtcagctccggcgacgccggctacggcgtggagtacccggcctcgtcccactacgtgaccgccgtcggcggcacctcgctgcgcaccgcgagcaccagccgcggctggagcgagaccgcgtggagcggcgcgggcagtggctgctcggcctacaacaccgcgctgtccggccagtccggcctcaccggctgctcccggcgcgccgtcgccgacgtctccgccgtggccgacccggccaccggcgtcgccgtctacgacagcacggcctaccagggccagagcggctggatggtcttcggcggcaccagcgtcgccgcaccgatcatcggtggcgtgtacggcctcgccgccaacgccgcgagcatcgacaacaactacccctacgcccacaccagctcgctcttcgacgtcacgtcgggcagcaacggcacctgcaccaccaccaagtggtgcaccgccggcaccggctgggacggccccaccggcctcggaacgccgaacggcaccggagccttcggacaccaccaccaccaccactga

SEQ ID NO: 7 - DNA, 5789 bp, Artificial Sequence
  empty pIJ86 vector
  unsure 2579..2582: /note=“n can be any of A, T, G, C”
gccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcgacctgcaggtcgactctagaggatctggggaattcgagctcggtaccagcccgacccgagcacgcgccggcacgcctggtcgatgtcggaccggagttcgaggtacgcggcttgcaggtccaggaaggggacgtccatgcgagtgtccgttcgagtggcggcttgcgcccgatgctagtcgcggttgatcggcgatcgcaggtgcacgcggtcgatcttgacggctggcgagaggtgcggggaggatctgaccgacgcggtccacacgtggcaccgcgatgctgttgtgggcacaatcgtgccggttggtaggatcctctagagtcgaccggcatgcaagcttggctgcagatctgatccgcggggcgccggcacttcgtgctggcgccccgcccccacccaccaggagaccgaccatgaccgacttcgacggacgcctgaccgaggggaccgtgaacctggtccaggaccccaacggcggtggctggtccgcccactgcgctgagcccggttgcgactgggccgacttcgccggaccgctcggcttccagggcctcgtggccatcgctcgccgacacacgcactgaccgcacgtcaaagccccgccggatcaccggcggggctctcttcggccctccaagtcacaccagccccaaggggcgtcgggagtggcggagggaacctctggcccgattggtgccaggattcccaccagaccaaagagcaacgggccggacttcgcacctccgacccgtccgctcccagactcgcgccccttagccgggcgagacaggaacgttgctcgtgcccagagtacggagcgatgccgaggcattgccagatcggcccgccgggccccgctgccactgcgggaccgcaattgcccacacaccgggcaaacggccgcgtatctactgctcagaccgctgccggatggcagcgaagcgggcgatcgcgcgtgtgacgcgagatgccgcccgaggcaaaagcgaacaccttgggaaagaaacaacagagtttcccgcacccctccgacctgcggtttctccggacggggtggatggggagagcccgagaggcgacagcctctgggaagtaggaagcacgtcgcggaccgaggctgcccgactgcggaaagccgcccggtacagccgccgccggacgctgtggcggatcagcggggacgccgcgtgcaagggctgcggccgcgccctgatggaccctgcctccggcgtgatcgtcgcccagacggcggccggaacgtccgtggtcctgggcctgatgcggtgcgggcggatctggctctgcccggtctgcgccgccacgatccggcacaagcgggccgaggagatcaccgccgccgtggtcgagtggatcaagcgcggggggaccgcctacctggtcaccttcacggcccgccatgggcacacggaccggctcgcggacctcatggacgccctccagggcacccggaagacgccggacagcccccggcggccgggcgcctaccagcgactgatcacgggcggcacgtgggccggacgccgggccaaggacgggcaccgggccgccgaccgcgagggcatccgagaccggatcgggtacgtcggcatgatccgcgcgaccgaagtcaccgtggggcagatcaacggctggcacccgcacatccacgcgatcgtcctggtcggcggccggaccgagggggagcggtccgcgaagcagatcgtcgccaccttcgagccgaccggcgccgcgctcgacgagtggcaggggcactggcggtccgtgtggaccgccgccctgcgcaaggtcaaccccgccttcacgcccgacgaccggcacggcgtcgacttcaagcggctggagaccgagcgcgacgccaacgacctcgccgagtacatcgccaagacccaggacgggaaggcgcccgccctcgaactcgcccgcgccgacctcaagacggcgaccggcgggaacgtcgccccgttcgaactcctcggacggatcggggacctgaccggcggcatgaccgaggacgacgccgccggggtcggctcgctggagtggaacctctcgcgctggcacgagtacgagcgggcaacccggggacgccgggccatcgaatggacccgctacctgcggcagatgctcgggctcgacggcggcgacaccgaggccgacgacctcgatctgctcctggcggccgacgccgacggcggggagctgcgggccggggtcgccgtgaccgaggacggatggcacgcggtcacccgccgcgccctcgacctcgaggcgacccgggccgccgaaggcaaggacggcaacgagnnnncggcggccgtgggcgaacgggtgcgggaggtcctggcgctggccgacgcggccgacacagtggtggtgctcacggcgggggaggtggccgaggcgtacgccgacatgctcgccgccctcgcccagcgccgcgaggaagcaactgcacgccgacggcgagagcaggacgacgaccaggacgacgacgccgacgaccgccaggagcgggccgcccggcacatcgcccggctcgcaagtgggcccacttcgcactaactcgctcccccccgccgtacgtcatcccggtgacgtacggcgggggtcggtgacgtacgcggcgacggcggccggggtcgaagccgcggccgcgcgcgatatcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggttcatgtgcagctccatcagcaaaaggggatgataagtttatcaccaccgactatttgcaacagcgccggtgatcgtgctatgatcgactgatgtcatcagcggtggagtgcaatgtcgtgcaatacgaatggcgaaaagccgagctcatcggtcagcttctcaaccttggggttacccccggcggtgtgctgctggtccacagctccttccgtagcgtccggcccctcgaagatgggccacttggactgatcgaggccctgcgtgctgcgctgggtccgggagggacgctcgtcatgccctcgtggtcaggtctggacgacgagccgttcgatcctgccacgtcgcccgttacaccggaccttggagttgtctctgacacattctggcgcctgccaaatgtaaagcgcagcgcccatccatttgcctttgcggcagcggggccacaggcagagcagatcatctctgatccattgcccctgccacctcactcgcctgcaagcccggtcgcccgtgtccatgaactcgatgggcaggtacttctcctcggcgtgggacacgatgccaacacgacgctgcatcttgccgagttgatggcaaaggttccctatggggtgccgagacactgcaccattcttcaggatggcaagttggtacgcgtcgattatctcgagaatgaccactgctgtgagcgctttgccttggcggacaggtggctcaaggagaagagccttcagaaggaaggtccagtcggtcatgcctttgctcggttgatccgctcccgcgacattgtggcgacagccctgggtcaactgggccgagatccgttgatcttcctgcatccgccagaggcgggatgcgaagaatgcgatgccgctcgccagtcgattggctgagctcatgagcggagaacgagatgacgttggaggggcaaggtcgcgctgattgctggggcaacacgtggagcggatcggggattgtctttcttcagctcgctgatgatatgctgacgctcaatgccgtttggcctccgactaacgaaaatcccgcatttggacggctgatccgattggcacggcggacggcgaatggcggagcagacgctcgtccgggggcaatgagatatgaaaaagcctgaactcaccgcgacgtatcgggccctggccagctagctagagtcgacctgcaggtccccggggatcggtcttgccttgctcgtcggtgatgtacttcaccagcttcgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccg

SEQ ID NO: 8 - DNA, 6986 bp, Artificial Sequence
  recombinant pIJ86 expression vector
  unsure 3776..3779: /note=“n can be any of A, T, G, C”
gccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcgacctgcaggtcgactctagaggatctggggaattcgagctcggtaccagcccgacccgagcacgcgccggcacgcctggtcgatgtcggaccggagttcgaggtacgcggcttgcaggtccaggaaggggacgtccatgcgagtgtccgttcgagtggcggcttgcgcccgatgctagtcgcggttgatcggcgatcgcaggtgcacgcggtcgatcttgacggctggcgagaggtgcggggaggatctgaccgacgcggtccacacgtggcaccgcgatgctgttgtgggcacaatcgtgccggttggtaggatcaatgtcacgacgcgtgaccgggaccatactgggcgggttgatcctcgccatggtccccttcctttccaccgcggccaacgccgcaccccaggccgcgccggcttccgtctcccacccgttccaccactcctgcgccacggtgaagccgggtcgggcgagctgcaatgccctcgtacgcagcgacatcgcccagagcgcggcgaccctcgcgcaccaagcggccgccccatccgggctctcgccggccaacctgcagagcgcctacaagctgccgtcctccacggccggatccggccagaccgtcgcgatcgtcgacgcctatgacgccccgaccgccgaagcggacttgaacgtgtaccgaagccagttcggactcggcgcgtgcacgaccgccaacggctgtttcaagaaggtcgaccagaacggcggcacgtcctatccgaggaaggacggcggctgggcgcaggagatctccctggacctcgacatggtctccgcggtctgccccaactgcaagatcgttctcgtcgaggcgaagaccaactcgttcgccaacctgggtaccgccgagaacaccgcggcgagtctcgcgaacgtcatcagcaacagctacggcggctcggacgcctctgacgcgagctatggctcgtactacaaccacccgggcaaggccatcacggtcagctccggcgacgccggctacggcgtggagtacccggcctcgtcccactacgtgaccgccgtcggcggcacctcgctgcgcaccgcgagcaccagccgcggctggagcgagaccgcgtggagcggcgcgggcagtggctgctcggcctacaacaccgcgctgtccggccagtccggcctcaccggctgctcccggcgcgccgtcgccgacgtctccgccgtggccgacccggccaccggcgtcgccgtctacgacagcacggcctaccagggccagagcggctggatggtcttcggcggcaccagcgtcgccgcaccgatcatcggtggcgtgtacggcctcgccgccaacgccgcgagcatcgacaacaactacccctacgcccacaccagctcgctcttcgacgtcacgtcgggcagcaacggcacctgcaccaccaccaagtggtgcaccgccggcaccggctgggacggccccaccggcctcggaacgccgaacggcaccggagccttcggacaccaccaccaccaccactgaagcttggctgcagatctgatccgcggggcgccggcacttcgtgctggcgccccgcccccacccaccaggagaccgaccatgaccgacttcgacggacgcctgaccgaggggaccgtgaacctggtccaggaccccaacggcggtggctggtccgcccactgcgctgagcccggttgcgactgggccgacttcgccggaccgctcggcttccagggcctcgtggccatcgctcgccgacacacgcactgaccgcacgtcaaagccccgccggatcaccggcggggctctcttcggccctccaagtcacaccagccccaaggggcgtcgggagtggcggagggaacctctggcccgattggtgccaggattcccaccagaccaaagagcaacgggccggacttcgcacctccgacccgtccgctcccagactcgcgccccttagccgggcgagacaggaacgttgctcgtgcccagagtacggagcgatgccgaggcattgccagatcggcccgccgggccccgctgccactgcgggaccgcaattgcccacacaccgggcaaacggccgcgtatctactgctcagaccgctgccggatggcagcgaagcgggcgatcgcgcgtgtgacgcgagatgccgcccgaggcaaaagcgaacaccttgggaaagaaacaacagagtttcccgcacccctccgacctgcggtttctccggacggggtggatggggagagcccgagaggcgacagcctctgggaagtaggaagcacgtcgcggaccgaggctgcccgactgcggaaagccgcccggtacagccgccgccggacgctgtggcggatcagcggggacgccgcgtgcaagggctgcggccgcgccctgatggaccctgcctccggcgtgatcgtcgcccagacggcggccggaacgtccgtggtcctgggcctgatgcggtgcgggcggatctggctctgcccggtctgcgccgccacgatccggcacaagcgggccgaggagatcaccgccgccgtggtcgagtggatcaagcgcggggggaccgcctacctggtcaccttcacggcccgccatgggcacacggaccggctcgcggacctcatggacgccctccagggcacccggaagacgccggacagcccccggcggccgggcgcctaccagcgactgatcacgggcggcacgtgggccggacgccgggccaaggacgggcaccgggccgccgaccgcgagggcatccgagaccggatcgggtacgtcggcatgatccgcgcgaccgaagtcaccgtggggcagatcaacggctggcacccgcacatccacgcgatcgtcctggtcggcggccggaccgagggggagcggtccgcgaagcagatcgtcgccaccttcgagccgaccggcgccgcgctcgacgagtggcaggggcactggcggtccgtgtggaccgccgccctgcgcaaggtcaaccccgccttcacgcccgacgaccggcacggcgtcgacttcaagcggctggagaccgagcgcgacgccaacgacctcgccgagtacatcgccaagacccaggacgggaaggcgcccgccctcgaactcgcccgcgccgacctcaagacggcgaccggcgggaacgtcgccccgttcgaactcctcggacggatcggggacctgaccggcggcatgaccgaggacgacgccgccggggtcggctcgctggagtggaacctctcgcgctggcacgagtacgagcgggcaacccggggacgccgggccatcgaatggacccgctacctgcggcagatgctcgggctcgacggcggcgacaccgaggccgacgacctcgatctgctcctggcggccgacgccgacggcggggagctgcgggccggggtcgccgtgaccgaggacggatggcacgcggtcacccgccgcgccctcgacctcgaggcgacccgggccgccgaaggcaaggacggcaacgagnnnncggcggccgtgggcgaacgggtgcgggaggtcctggcgctggccgacgcggccgacacagtggtggtgctcacggcgggggaggtggccgaggcgtacgccgacatgctcgccgccctcgcccagcgccgcgaggaagcaactgcacgccgacggcgagagcaggacgacgaccaggacgacgacgccgacgaccgccaggagcgggccgcccggcacatcgcccggctcgcaagtgggcccacttcgcactaactcgctcccccccgccgtacgtcatcccggtgacgtacggcgggggtcggtgacgtacgcggcgacggcggccggggtcgaagccgcggccgcgcgcgatatcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggttcatgtgcagctccatcagcaaaaggggatgataagtttatcaccaccgactatttgcaacagcgccggtgatcgtgctatgatcgactgatgtcatcagcggtggagtgcaatgtcgtgcaatacgaatggcgaaaagccgagctcatcggtcagcttctcaaccttggggttacccccggcggtgtgctgctggtccacagctccttccgtagcgtccggcccctcgaagatgggccacttggactgatcgaggccctgcgtgctgcgctgggtccgggagggacgctcgtcatgccctcgtggtcaggtctggacgacgagccgttcgatcctgccacgtcgcccgttacaccggaccttggagttgtctctgacacattctggcgcctgccaaatgtaaagcgcagcgcccatccatttgcctttgcggcagcggggccacaggcagagcagatcatctctgatccattgcccctgccacctcactcgcctgcaagcccggtcgcccgtgtccatgaactcgatgggcaggtacttctcctcggcgtgggacacgatgccaacacgacgctgcatcttgccgagttgatggcaaaggttccctatggggtgccgagacactgcaccattcttcaggatggcaagttggtacgcgtcgattatctcgagaatgaccactgctgtgagcgctttgccttggcggacaggtggctcaaggagaagagccttcagaaggaaggtccagtcggtcatgcctttgctcggttgatccgctcccgcgacattgtggcgacagccctgggtcaactgggccgagatccgttgatcttcctgcatccgccagaggcgggatgcgaagaatgcgatgccgctcgccagtcgattggctgagctcatgagcggagaacgagatgacgttggaggggcaaggtcgcgctgattgctggggcaacacgtggagcggatcggggattgtctttcttcagctcgctgatgatatgctgacgctcaatgccgtttggcctccgactaacgaaaatcccgcatttggacggctgatccgattggcacggcggacggcgaatggcggagcagacgctcgtccgggggcaatgagatatgaaaaagcctgaactcaccgcgacgtatcgggccctggccagctagctagagtcgacctgcaggtccccggggatcggtcttgccttgctcgtcggtgatgtacttcaccagcttcgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccg

SEQ ID NO: 9 - DNA, 28 bp, Artificial Sequence
  PCR oligonucleotide primer
aaatgatcaatgtcacgacgcgtgaccg

SEQ ID NO: 10 - DNA, 47 bp, Artificial Sequence
  PCR oligonucleotide primers
aaaaagcttcagtggtggtggtggtggtgtccgaaggctccggtgcc

SEQ ID NO: 11 - DNA, 1235 bp, Artificial Sequence
  amplicon
aaatgatcaatgtcacgacgcgtgaccgggaccatactgggcgggttgatcctcgccatggtccccttcctttccaccgcggccaacgccgcaccccaggccgcgccggcttccgtctcccacccgttccaccactcctgcgccacggtgaagccgggtcgggcgagctgcaatgccctcgtacgcagcgacatcgcccagagcgcggcgaccctcgcgcaccaagcggccgccccatccgggctctcgccggccaacctgcagagcgcctacaagctgccgtcctccacggccggatccggccagaccgtcgcgatcgtcgacgcctatgacgccccgaccgccgaagcggacttgaacgtgtaccgaagccagttcggactcggcgcgtgcacgaccgccaacggctgtttcaagaaggtcgaccagaacggcggcacgtcctatccgaggaaggacggcggctgggcgcaggagatctccctggacctcgacatggtctccgcggtctgccccaactgcaagatcgttctcgtcgaggcgaagaccaactcgttcgccaacctgggtaccgccgagaacaccgcggcgagtctcgcgaacgtcatcagcaacagctacggcggctcggacgcctctgacgcgagctatggctcgtactacaaccacccgggcaaggccatcacggtcagctccggcgacgccggctacggcgtggagtacccggcctcgtcccactacgtgaccgccgtcggcggcacctcgctgcgcaccgcgagcaccagccgcggctggagcgagaccgcgtggagcggcgcgggcagtggctgctcggcctacaacaccgcgctgtccggccagtccggcctcaccggctgctcccggcgcgccgtcgccgacgtctccgccgtggccgacccggccaccggcgtcgccgtctacgacagcacggcctaccagggccagagcggctggatggtcttcggcggcaccagcgtcgccgcaccgatcatcggtggcgtgtacggcctcgccgccaacgccgcgagcatcgacaacaactacccctacgcccacaccagctcgctcttcgacgtcacgtcgggcagcaacggcacctgcaccaccaccaagtggtgcaccgccggcaccggctgggacggccccaccggcctcggaacgccgaacggcaccggagccttcggacaccaccaccaccaccactgaagcttttt

SEQ ID NO: 12 - protein, 33 aa, Triticeae
LQLQPFPQPQLPYPQPQLPYPQPQLPYPQPQPF

SEQ ID NO: 13 - DNA, 1395 bp, Saccharomyces cerevisiae
tctagaatgagattcccatctattttcaccgctgatttgtttgctgcttcttctgctttggctgctccagttaagactactactgaagatgaaactgctcaaattccagctgaagctgttattggttactctgatttggaaggtgatttcgatgttgctgttttgccattctctaactctaccaacaatggtttgttgttcatcaacaccaccattgcttctattgctgctaaagaagaaggtgtctctttggaaaagagagaaactgaagccgctcctcaagcagctccagcatctgtttctcatccatttcatcattcttgtgctacagttaaacctggtagagcatcttgtaatgctttagttagatccgatattgctcaatctgcagctacattagcacatcaagccgcagctccatctggtttgtctcctgctaacttgcagtctgcatacaaattgccatcttctacagctggttctggtcaaactgttgctatagttgatgcttatgacgctcctacagctgaagctgatttgaacgtttacagatctcagtttggtttaggtgcttgtactacagctaatggttgcttcaaaaaggtcgatcagaacggtggtacttcttacccaagaaaagatggaggttgggctcaagaaatttccttggatttggacatggtttctgctgtttgtcctaactgcaagatcgttttagttgaagctaagactaactcctttgctaatttaggtacagctgaaaacactgcagcttcattagctaacgttatctctaattcttacggaggttctgatgcatcagacgcttcttatggttcttactacaatcatccaggtaaagccattactgtttcttcaggtgacgcaggttatggtgttgaatacccagcttcttctcattacgttacagctgttggtggtacatctttgagaactgcttctacttcaagaggttggtctgagacagcttggtctggtgctggttcaggttgttctgcatataatactgctttatcaggtcaatcaggtttgactggttgttctcgtagagcagttgctgatgtttctgcagttgctgatcctgctactggtgttgctgtttatgattctactgcttaccaaggtcaaagtggttggatggtttttggtggaacttctgttgcagctccaattattggtggtgtttatggtttggctgcaaacgcagcttctattgataacaattatccatacgctcacacctcgtctttgttcgatgttacatctggttctaacggtacatgtacaactactaagtggtgtactgctggtactggttgggatggtccaacaggtttaggtactccaaatggtacaggtgctttttaaaagctt

SEQ ID NO: 14 - DNA, 1257 bp, Saccharomyces cerevisiae
tctagaatgagattcccatctattttcaccgctgatttgtttgctgcttcttctgctttggctgctccagttaagactactactgaagatgaaactgctcaaattccagctgaagctgttattggttactctgatttggaaggtgatttcgatgttgctgttttgccattctctaactctaccaacaatggtttgttgttcatcaacaccaccattgcttctattgctgctaaagaagaaggtgtctctttggaaaagagagaaactgaagccgcagctccatctggtttgtctcctgctaacttgcagtctgcatacaaattgccatcttctacagctggttctggtcaaactgttgctatagttgatgcttatgacgctcctacagctgaagctgatttgaacgtttacagatctcagtttggtttaggtgcttgtactacagctaatggttgcttcaaaaaggtcgatcagaacggtggtacttcttacccaagaaaagatggaggttgggctcaagaaatttccttggatttggacatggtttctgctgtttgtcctaactgcaagatcgttttagttgaagctaagactaactcctttgctaatttaggtacagctgaaaacactgcagcttcattagctaacgttatctctaattcttacggaggttctgatgcatcagacgcttcttatggttcttactacaatcatccaggtaaagccattactgtttcttcaggtgacgcaggttatggtgttgaatacccagcttcttctcattacgttacagctgttggtggtacatctttgagaactgcttctacttcaagaggttggtctgagacagcttggtctggtgctggttcaggttgttctgcatataatactgctttatcaggtcaatcaggtttgactggttgttctcgtagagcagttgctgatgtttctgcagttgctgatcctgctactggtgttgctgtttatgattctactgcttaccaaggtcaaagtggttggatggtttttggtggaacttctgttgcagctccaattattggtggtgtttatggtttggctgcaaacgcagcttctattgataacaattatccatacgctcacacctcgtctttgttcgatgttacatctggttctaacggtacatgtacaactactaagtggtgtactgctggtactggttgggatggtccaacaggtttaggtactccaaatggtacaggtgctttttaaaagctt